(19) United States
(12) Patent Application Publication
(10) Pub. No.: US 2022/0380425 A1
(43) Pub. Date: Dec. 1, 2022

(54) ANGPTL3 BASED VACCINE FOR THE TREATMENT OF LIVER DISEASE

(71) Applicant: CADILA HEALTHCARE LIMITED, Ahmedabad, Gujarat (IN)

(72) Inventors: Mukul JAIN, Ahmedabad, Gujarat (IN); Suresh GIRI, Ahmedabad, Gujarat (IN); Rajesh BAHEKAR, Ahmedabad, Gujarat (IN); Rajendra CHOPADE, Ahmedabad, Gujarat (IN); Debdutta BANDYOPADHYAY, Ahmedabad, Gujarat (IN)

(21) Appl. No.: 17/624,114

(22) PCT Filed: Jul. 4, 2020
(86) PCT No.: PCT/IB2020/056309

§ 371 Date: Dec. 30, 2021

(30) Foreign Application Priority Data
Oct. 16, 2019 (IN) 201921041918
Jul. 4, 2020 (IN) 201921026805

(51) Int. Cl. C07K 14/515; A61P 1/16; A61K 39/00

(52) U.S. Cl. CPC C07K 14/515; A61P 1/16; A61K 39/0005

(57) ABSTRACT

The present invention relates to a vaccine capable to induce the formation of antibodies directed to angiopoietin-like 3 in vivo. More specifically, the present invention relates to a use of a vaccines which are able to influence the angiopoietin-like 3 mediated immune response for the treatment of liver diseases such as non-alcoholic steatohepatitis and non-alcoholic fatty liver disease and hyperlipidaemia, hypercholesterolemia, or atherosclerosis including the complications lead to the cardiovascular diseases (CVD) which causes morbidity and mortality.

### FIELD OF THE INVENTION

The present invention relates to novel peptide(s), their enantiomers, their diastereoisomers, their stereoisomers, their pharmaceutically acceptable salts or their prodrugs. Said peptide(s) may further be conjugated with suitable immunogenic carrier to prepare a vaccine which is capable to induce the formation of antibodies directed to angiopoietin-like 3 protein. The present invention also relates to a use of vaccines which are able to influence the angiopoietin-like 3 mediated immune response for the treatment of liver diseases, hyperlipidaemia, hypercholesterolemia, or atherosclerosis including the complications which lead to cardiovascular diseases (CVD) and thereby causes morbidity and mortality. The present invention also discloses the use of the conjugated peptides of the present invention as vaccines suitable to influence the angiopoietin-like 3 mediated immune response for the treatment of liver diseases, hyperlipidaemia, hypercholesterolemia, or atherosclerosis including the complications which lead to cardiovascular diseases (CVD) and thereby causes morbidity and mortality.

### BACKGROUND OF THE INVENTION

Angiopoietin-like 3 (referred herein as ANGPTL3 or hANGPTL3) is an angiopoietin protein encoded by the human angiopoietin-like 3 gene that is reported to be involved in regulating lipid metabolism. ANGPTL3 is known to be primarily produced in hepatocytes in humans, and after synthesis is secreted into circulation. ANGPTL3 acts as an inhibitor of lipoprotein lipase, which catalyzes hydrolysis of triglycerides, and endothelial lipase, which hydrolyzes lipoprotein phospholipids. Inhibition of these enzymes can cause increases in plasma levels of triglycerides, high-density lipoproteins (HDL), and phospholipids. ANGPTL3 is a 460-amino acid polypeptide that consists of a signal peptide, N-terminal coiled-coil domain, and a C-terminal fibrinogen (FBN)—like domain. Further, loss-of-function mutations in ANGPTL3 lead to familial hypobetalipoproteinemia, which is characterized by low levels of triglycerides and low-density lipoprotein (LDL-C) in plasma. In humans, loss-of-function in ANGPTL3 is also correlated with a decreased risk of atherosclerotic cardiovascular disease. An effective therapeutic that targets ANGPTL3 could provide a beneficial impact in the treatment (including prophylactic treatment) of cardiometabolic diseases such as hypertriglyceridemia, obesity, hyperlipidemia, abnormal lipid and/or cholesterol metabolism, atherosclerosis, type II diabetes mellitus, cardiovascular disease, coronary artery disease, non-alcoholic steatohepatitis (NASH), non-alcoholic fatty liver disease (NAFLD), homozygous and heterozygous familial hypercholesterolemia, statin resistant hypercholesterolemia and other metabolic-related disorders and diseases. Antibodies targeting ANGPTL3 have been identified as being capable of blocking or neutralizing activity of ANGPTL3 by specifically binding with ANGPTL3 protein (see, e.g., International Patent Application Publication Nos. WO2008/073300 and WO2012/174178). Certain double-stranded RNA-based compounds have been identified as being capable of inhibiting the expression of an ANGPTL3 gene (see, e.g., International Patent Application Publication Nos. WO 2012/177784, WO 2016/168286, WO 2016/154127 and WO2019/055633). Compounds comprising a modified oligonucleotide of 10 to 30 linked nucleosides in length targeted to ANGPTL3 have also been identified as being able to reduce ANGPTL3 expression in an animal (see, e.g., International Patent Application Publication Nos.

WO2011/085271 and WO2015/100394 and WO2015/168589).

ANGPTL3 also acts as dual inhibitor of lipoprotein lipase (LPL) and endothelial lipase (EL), thereby increasing plasma triglyceride, LDL cholesterol and HDL cholesterol in mice and humans. It is also reported to be a direct target gene of LXR and has role in lipid metabolism. Genetic, loss-of-function variants in LPL have been shown to increase the risk of coronary artery disease, and gain-of function variants have been shown to decrease the risk (1, 2 and 3). Genetic and therapeutic antagonism of ANGPTL3 in humans and of ANGPTL3 in mice is associated with decreased levels of major lipid fractions (TG and LDL-C) and decreased odds of atherosclerotic cardiovascular disease (4). ANGPTL3 deficiency is associated with a reduced risk of CAD (5). In humans, Evinacumab (a therapeutic mAb developed against ANGPTL3) caused a dose-dependent reduction in fasting triglyceride levels of up to 76% and LDL cholesterol levels of up to 23%. (4) Antisense oligonucleotides (ASO for ANGPTL3) showed 85% reduction in liver triglyceride levels, and improvement is liver steatosis. (6) ANGPTL3 levels were significantly higher in patients with definite NASH (P<0.05) and borderline NASH compared with controls (7). There is significant positive association observed between changes in ANGPTL3 and AST and CK18 (8).

Thus, several compounds reducing the amount of circulating ANGPTL3 or neutralizing its activity are being tested pre-clinically and clinically (such as monoclonal antibodies or antisense-oligonucleotides). However, said publications do not demonstrate or explicitly suggest a vaccine capable to induce the formation of antibodies directed to angiopoietin-like 3 (ANGPTL3) in vivo. While, the current invention provides novel peptide(s) and use of said peptide(s) to prepare a vaccine(s) which are able to influence the angiopoietin-like 3 (ANGPTL3) for the treatment of liver disease.

### SUMMARY OF THE INVENTION

The present invention provides novel ANGPTL3 based peptide sequence(s) optionally conjugated with a suitable immunogenic carrier and is capable to induce auto anti-ANGPTL3 antibodies. Said peptide(s) are used to prepare vaccine according to the present invention. In one of the aspects, the present invention provides ANGPTL3 based vaccine for the treatment of liver diseases, hyperlipidaemia, hypercholesterolemia, or atherosclerosis including the complications that lead to cardiovascular diseases (CVD) which causes morbidity and mortality. The said vaccine is preferably peptide based vaccine. The present invention also relates to the use of a vaccine for the manufacture of the medicament for the treatment of liver diseases, hyperlipidaemia, hypercholesterolemia, or atherosclerosis including the complications lead to the cardiovascular diseases (CVD) which causes morbidity and mortality, preferably for the treatment of liver diseases, more preferably for the treatment of NASH and NAFLD. In a further aspect, current invention provides method of screening of a peptide based compounds capable to induce the formation of antibodies directed to angiopoietin-like 3 (ANGPTL3) in vivo. Such compounds can be an antigenic ANGPLTL3 peptide. In further more aspect, the present invention provides stable vaccine composition comprising a peptide of at least 2 to about 50 amino acids and an immunogenic carrier. Such vaccine compositions according to the present invention can be used for the prevention and/or treatment of ANGPTL3-related health disorders or diseases such as liver diseases, hyperlipidaemia, hypercholesterolemia, or atherosclerosis including the complications that lead to cardiovascular diseases (CVD) which causes morbidity and mortality, preferably for the prevention and/or treatment of liver diseases, more preferably for the prevention and/or treatment of NASH and NAFLD.

### BRIEF DESCRIPTION OF FIGURES

FIG. 1 depicts examples of some of the protected amino acids used in Fmoc based-Solid Phase Peptide Synthesis (SPPS) of peptides.

FIG. 2 depicts conjugation of peptide (SEQ ID NO.: 1) with diphtheria toxoid for vaccine preparation

FIG. 3 depicts conjugation of peptide (SEQ ID NO.: 87) with diphtheria toxoid for vaccine preparation

### LIST OF ABBREVIATIONS USED HEREIN IN THE PRESENT APPLICATION

μl: microliter
2F-Phe: 2-fluorophenylalanine
2-Pal: 2-Pyridylalanine
2-Thi: (2-Thienyl)-alanine
4-Pal: 4-Pyridylalanine
Ab: antibody
Abu (CN): 2-amino-4-cyanobutanoic acid
Ac: Acetyl
AC₃C—OH: 1-aminocyclopropane-1-carboxylic acid
AC₅C—OH: 1-aminocyclopentane-1-carboxylic acid
AC₆C—OH: 1-aminocyclohexane-1-carboxylic acid
ACN: Acetonitrile
Aib: α-Amino-isobutyric acid
ANGPTL3: Angiopoietin-like 3
ANGPTL4: Angiopoietin-like 4
APPA: 2-Aminophenyl pentanoic acid
Arg(NO₂): Arginine (NO₂)
Boc: tert-Butoxycarbonyl
But: O-tert-butyl group
Cit: Citrulline,
DCM: Dichloromethane
DIPCDI: Di-isopropylcarbodiimide
DIPEA: Diisopropylethylamine
DMF: N, N-Dimethylformamide
DT: diphtheria toxin
Et: Ethyl
Et₂O: Diethyl ether
Fmoc: Fluorenylmethoxycarbonyl
g: Gram (s)
h: Hour (s)
Har: homoarginine
HBTU: 2-(1H-benzotriazole-1-yl)-1, 1, 3, 3-tetramethyl aminium
hexafluorophosphate
HOAt: 7-Aza-hydroxybenzotriazole
HOBt: 1-Hydroxybenzotriazole
HoGlu or Homo-Glu: homoglutamic acid
HoLeu: homoleucine
HoSer: homoserine
HPLC: High Performance Liquid Chromatography
K(Biotin): Lysine(Biotin)
KLH: keyhole limpet haemocyanin
L: Liter
LC/MS: Liquid Chromatography/Mass Spectrometry
LPL: Lipoprotein Lipase
MCS: 6-maleimido caproic acyl N-hydroxysuccinimide ester
Me: Methyl
mg: milligram (s)
Min: minute (s)
mL: milliliter
mmol: millimole (s)
MS: Mass Spectrometry
NAFLD: non-alcoholic fatty liver disease
NASH: non-alcoholic steatohepatitis
Nle: Norleucine
NMe-Ile: N-methyl-isoleucine
NMe-Leu: N-methyl-leucine
NMe-Nle: N-methyl-norleucine
Nva: Norvaline
OMe-HoSer or HoSer(OMe): O-methyl-homoserine
OMe-Ser or Ser(OMe): O-methyl-serine
OMe-Thr or Thr(OMe): O-methyl-threonine
Orn: Ornithine
pAb: Polyclonal antibody
Pal: 3-Pyridylalanine
PyBOP: Benzotriazole-1-yl-oxy-tris-pyrrolidino-phosphonium
hexafluorophosphate
RU: Resonance unit
Sar: Sarcosine
SE1: specific epitope 1
SPPS: Solid Phase Peptide Synthesis
TBTU: 2-(1H-benzotriazole-1-yl)-1, 1, 3, 3-tetramethylaminium tetrafluoroborate
TFA: Trifluoroacetic acid
Tha: (4-Thiazolyl)-alanine
TIPS: Triisopropylsilane
TMS: Trimethylsilyl
Trt: Trityl group
TT: tetanus toxoid
VLP: Virus-like particle
α-Me-APPA: alpha-methyl-2-aminophenyl pentanoic acid
α-Me-2,6-diF-Phe: alpha-methyl-2,6-diflurophenylalanine
α-Me-2F-Phe: alpha-methyl-2-fluorophenylalanine
α-Me-Asp: alpha-methyl-aspartic acid
α-Me-E or αMe-Glu: alpha-methyl-glutamic acid
α-Me-L: alpha-methyl-leucine
α-Me-Phe: alpha-methyl-phenylalanine
α-Me-Pro: alpha-methyl-proline
β-Ala: beta alanine

Abbreviations of natural amino acids as used in the current application are provided in below table.

| | Abbreviation | Abbreviation |
|---|---|---|
| Full Name | (3 Letter) | (1 Letter) |
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### Definitions

The term “Animal” as used herein, refers to a human or non-human animal, including, but not limited to, mice, rats, rabbits, dogs, cats, pigs, and non-human primates, including, but not limited to, monkeys and chimpanzees.

The term “antigenic ANGPTL3 peptide”, as used herein, refers to the peptide which has an ability to induce auto anti-ANGPTL3 antibodies in an animal tested or a patient administered with the said peptide.

The term “ANGPTL3-related health disorders or diseases”, as used herein, refers to disorders or diseases wherein expression of ANGPTL3 is detrimental to health of the animal.

The term “fragment” as used herein refers to portion of the mentioned peptides or any other antigenic peptide which is capable to induce the formation of antibodies directed to angiopoietin-like 3.

The term “liver disease” is used herein in the broadest sense and refers to any disease of the liver associated with any type of liver injury, regardless of the underlying cause, herein to refer to liver diseases such as fatty liver, Nonalcoholic fatty liver disease (NAFLD) or nonalcoholic steatohepatitis (NASH) and includes, without limitation, inflammatory diseases of the liver and liver tumors. Inflammatory diseases of the liver include, for example, cirrhosis, such as, alcoholic liver cirrhosis and primary biliary cirrhosis (PBC), liver fibrosis, chronic hepatitis, i.e. chronic autoimmune hepatitis, chronic alcoholic hepatitis.

An “effective amount” of an antigenic peptide of the invention, or composition thereof, is an amount that is delivered to a mammalian subject, either in a single dose or as part of a series, which is effective for inducing an immune response against target antigen in said subject. This amount varies depending upon the health and physical condition of the individual to be treated, the taxonomic group of individual to be treated, the capacity of the individual's immune system to synthesize antibodies, the formulation of the vaccine, and other relevant factors. It is expected that the amount will fall in a relatively broad range that can be determined through routine trials.

The term “immunogenic carrier” as used herein, refers to materials which have the property of independently eliciting an immunogenic response in a host animal and which can be covalently coupled to a peptide, polypeptide or protein either directly via formation of peptide or ester bonds between free carboxyl, amino or hydroxyl groups in the peptide, polypeptide or protein and corresponding groups on the immunogenic carrier material, or alternatively by bonding through a conventional bifunctional linking group, or as a fusion protein.

A “pharmaceutically effective dose” or “therapeutically effective dose” is that dose required to treat or prevent, or alleviate one or more ANGPTL3 related disorder or symptom in a subject, preferably in the present invention, for NASH or NAFLD. The pharmaceutically effective dose depends on inter alia the specific compound to administer, the severity of the symptoms, the susceptibility of the subject to side effects, the type of disease, the composition used, the route of administration, the type of mammal being treated, the physical characteristics of the specific mammal under consideration such as health and physical condition, concurrent medication, the capacity of the individual's immune system to synthesize antibodies, the degree of protection desired, and other factors that those skilled in the medical arts will recognize. For prophylaxis purposes, the amount of peptide in each dose is selected as an amount which induces an immunoprotective response without significant adverse side effects in typical vaccines. Following an initial vaccination, subjects may receive one or several booster immunisations adequately spaced.

The term “preventing”, as used herein, covers measures not only to prevent the occurrence of disease, such as risk factor reduction, but also to arrest its progress and reduce its consequences once established.

The term “treatment” includes the improvement and/or reversal of the symptoms of disease. A compound which causes an improvement in any parameter associated with disease when used in the screening methods of the instant invention may thereby be identified as a therapeutic compound. The term “treatment” refers to both therapeutic treatment and prophylactic or preventative measures. For example, those who may benefit from treatment with compositions and methods of the present invention include those already with a disease and/or disorder as well as those in which a disease and/or disorder is to be prevented (e.g., using a prophylactic treatment of the present invention).

The term “virus-like particle” as used herein, refers to a structure resembling a virus particle but which has been demonstrated to be non-pathogenic. In general, virus-like particles lack at least part of the viral genome. Also, virus-like particles can often be produced in large quantities by heterologous expression and can be easily purified. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, or RNA-phage.

The term “natural amino acids” indicates all twenty amino acids, which are present in nature. List of natural amino acids are given in the present application with their one-letter and three-later codes.

The term “unnatural amino acids” or “non-natural amino acids” preferably represents either replacement of L-amino acids with corresponding D-amino acids such as replacement of L-Ala with D-Ala and the like or suitable modifications of the L or D amino acids, amino alkyl acids, either by
α-alkylation such as substitution of Ala with α-methyl Ala (Aib), replacement of Leu with α-methyl Leu;
substitution on the side chain of amino acid such as substitution of aromatic amino acid side chain with halogen, (C1-C3) alkyl, aryl groups, more specifically the replacement of Phe with halo Phe;
β amino acids such as 3 alanine.

The various groups, radicals and substituents used anywhere in the specification are described in the following paragraphs.

The term “alkyl” used herein, either alone or in combination with other radicals, denotes a linear or branched radical containing one to eighteen carbons, such as methyl, ethyl, n-propyl, iso-propyl, n-butyl, sec-butyl, tert-butyl, amyl, t-amyl, n-pentyl, n-hexyl, iso-hexyl, heptyl, octyl, decyl, tetradecyl, octadecyl and the like.

The term “cycloalkyl” used herein, either alone or in combination with other radicals, denotes a radical containing three to seven carbons, such as cyclopropyl, cyclobutyl, cyclopentyl, cyclohexyl, cycloheptyl and the like.

Unless otherwise indicated, the term ‘amino acid’ as employed herein alone or as part of another group includes, without limitation, an amino group and a carboxyl group linked to the same carbon, referred to as ‘α’ carbon.

The absolute ‘S’ configuration at the ‘α’ carbon is commonly referred to as the ‘L’ or natural configuration. The ‘R’ configuration at the ‘α’ carbon is commonly referred to as the ‘D’ amino acid. In the case where both the ‘α-substituents’ is equal, such as hydrogen or methyl, the amino acid are Gly or Aib and are not chiral.

The term “derivative(s)” as used herein indicates substitution with non-natural amino acid(s), for example, homologous non-natural amino acid of respective amino acid.

While the invention has been primarily exemplified in relation to peptides, it is also be understood that the peptide linkage between the residues may be replaced by a non-peptide bond provided that the therapeutic potential is retained. The person skilled in the art is aware of such suitable modifications, such as thioamide bond formation, N-methylation of amide bonds and the like.

It is to be clearly understood that the compounds of the invention include peptide amides and non-amides and peptide analogues, including but not limited to the following:
a) Compounds in which one or more amino acid is replaced by its corresponding D-amino acid. The skilled person will be aware that retro-inverso amino acid sequences can be synthesized by standard methods (9);
b) Compounds, in which the peptide bond is replaced by a structure more resistant to metabolic degradation. (10) and
c) Compounds in which individual amino acid is replaced by analogous structures for example Ala with Aib; Lys with Cit.

Throughout the description the conventional one-letter and three-letter code for natural amino acids are used as well as generally acceptable three-letter codes for other unnatural amino acids such as Aib (α-amino isobutanoic acid) are used.

### EMBODIMENTS OF THE INVENTION

In one embodiment, the present invention provides novel peptide(s) of general formula (I) or their pharmaceutically acceptable salts.

In another embodiment, the novel peptide(s) of general formula (I) are conjugated with suitable immunogenic carrier(s) to prepare vaccine(s) which are able to induce the formation of antibodies which bind specifically to ANGPTL3 in living systems. The interaction of the antibodies with ANGPTL3 inhibits ANGPTL3, which serves as an inhibitor of lipoprotein lipase (LPL) and thereby reduces plasma triglyceride clearance.

In one of the embodiments, the present invention provides solvates of novel peptide(s) of general formula (I) or their pharmaceutically acceptable salts.

In another embodiment, the present invention provides novel intermediates involved in synthesis novel peptide(s) of general formula (I) or their pharmaceutically acceptable salts.

In one of the embodiments, the present invention provides suitable mixture(s) of novel peptide(s) of general formula (I) or their pharmaceutically acceptable salts or theirs solvates or their pharmaceutically acceptable salts.

In a further embodiment, the present invention provides pharmaceutical composition(s) containing peptide(s) of general formula (I) and/or their pharmaceutically acceptable salts, solvates and their mixtures in combination with media selected from pharmaceutically acceptable adjuvant(s), immunogenic carrier(s), solvent(s), diluent(s), excipient(s) and other media normally employed in their manufacture. The said pharmaceutical composition(s) or their combinations are suitable as vaccine(s) against ANGPTL3 gene.

In a still further embodiment, the present invention provides use of the novel peptide(s) of general formula (I) alone or when conjugated with suitable immunogenic carrier for the treatment or prevention of diseases mediating through ANGPTL3.

According to a particularly preferred embodiment of the present invention, the immunogenic carrier is selected from the group comprising of diphtheria toxin (DT), keyhole limpet haemocyanin (KLH), CRM (preferably CRM197), tetanus toxoid (TT), protein D or any other protein or peptide containing helper T-cell epitopes.

In a further preferred embodiment of the present invention, the immunogenic carrier is selected from the group consisting of diphtheria toxin (DT), keyhole limpet haemocyanin (KLH), CRM (preferably CRM197), tetanus toxoid (TT), protein D or any other protein or peptide containing helper T-cell epitopes and the peptide is conjugated with suitable linker(s) like 6-maleimido caproic acyl N-hydroxysuccinimide ester (MCS).

In a further preferred embodiment, the peptide(s) according to the present invention used for preparation of the vaccine of the present invention contains at its N- and/or C-terminus at least one cysteine residue bound directly or via a spacer sequence. This cysteine residue serves as a reactive group in order to bind the peptide to another molecule or a carrier protein.

In one of the preferred embodiment, the novel peptide(s) of general formula (I) or their pharmaceutically acceptable salt(s), solvate(s) are therapeutic compound(s) which can be used to treat ANGPTL3 related disorder. In a more preferred embodiments, the novel peptides of general formula (I) or their pharmaceutically acceptable salt(s), solvate(s) are therapeutically effective antigenic ANGPTL3 peptide.

In one of the preferred embodiments, the antigenic ANGPTL3 peptide is selected from signal peptide region of ANGPTL3 or its fragments thereof.

In another embodiment, the present invention provides antigenic ANGPTL3 peptide capable to induce the formation of antibodies directed to angiopoietin-like 3. Preferably, the antigenic ANGPTL3 peptide according to the present invention comprises 2 to 50 amino acid residues.

In such embodiment, the antigenic ANGPTL3 peptide according to the present invention comprises at least 2 amino acids, at least 3 amino acids, at least 4 amino acids, at least 5 amino acids, at least 6 amino acids, at least 7 amino acids, at least 8 amino acids, at least 9 amino acids, at least 10 amino acids, at least 11 amino acids, at least amino acids, at least 13 amino acids, at least 14 amino acids, at least 15 amino acids, at least 16 amino acids, at least 17 amino acids, at least 18 amino acids, at least 19 amino acids, at least 20 amino acids, at least 21 amino acids, at least 22 amino acids, at least 23 amino acids, at least 24 amino acids, at least 25 amino acids, at least 26 amino acids, at least 27 amino acids, at least 28 amino acids, at least 29 amino acids, at least 30 amino acids, at least 31 amino acids, at least 32 amino acids, at least 33 amino acids, at least 34 amino acids, at least 35 amino acids, at least 36 amino acids, at least 37 amino acids, at least 38 amino acids, at least 39 amino acids, at least 40 amino acids, at least 41 amino acids, at least 42 amino acids, at least 43 amino acids, at least 44 amino acids, at least 45 amino acids, at least 46 amino acids, at least 47 amino acids, at least 48 amino acids, at least 49 amino acids or at least 50 amino acids.

In one of the embodiments, the antigenic ANGPTL3 peptide according to the present invention comprises amino acids between 2 to 5 amino acid residues, 2 to 7 amino acid residues, 2 to 10 amino acid residues, 2 to 12 amino acid residues, 2 to 15 amino acid residues, 2 to 18 amino acid residues, 2 to 20 amino acid residues, 2 to 22 amino acid residues, 2 to 25 amino acid residues, 2 to 30 amino acid residues, 2 to 33 amino acid residues, 2 to 35 amino acid residues, 2 to 40 amino acid residues, 2 to 42 amino acid residues, 2 to 45 amino acid residues or 2 to 50 amino acid residues.

In a more preferred embodiment, the antigenic ANGPTL3 peptide according to the present invention comprises 5 to 50 amino acid residues.

In one of the embodiments, the present invention provides use of an antigenic ANGPTL3 peptide for the treatment of ANGPTL3-related health disorders. In a preferred embodiment, the present invention provides use of an antigenic ANGPTL3 peptide for the treatment of liver diseases, hyperlipidaemia, hypercholesterolemia, or atherosclerosis including the complications lead to the cardiovascular diseases (CVD) which causes morbidity and mortality. Preferably, the liver disease according to the present invention is selected from non-alcoholic steatohepatitis (NASH) and non-alcoholic fatty liver disease (NAFLD).

In one embodiment, the present invention provides use of a vaccine for the treatment of ANGPTL3-related health disorders. In a preferred embodiment, the present invention provides use of a vaccine for the treatment of liver disease wherein effect of ANGPTL3 is detrimental.

In a preferred embodiment, the present invention provides use of a vaccine for the treatment of liver diseases, hyperlipidaemia, hypercholesterolemia, or atherosclerosis including the complications lead to the cardiovascular diseases (CVD) which causes morbidity and mortality.

In a more preferred embodiment, the present invention provides use of a vaccine for the treatment of liver disease wherein liver disease is selected from non-alcoholic steatohepatitis (NASH) and non-alcoholic fatty liver disease or alcoholic liver disease (NAFLD).

In another embodiment, the present invention provides use of a vaccine for the manufacture of the medicament. The said vaccine can be used for the treatment of liver diseases, hyperlipidaemia, hypercholesterolemia, or atherosclerosis including the complications lead to the cardiovascular diseases (CVD) which causes morbidity and mortality, preferably for the treatment of non-alcoholic steatohepatitis (NASH) or non-alcoholic fatty liver disease (NAFLD). Preferably, vaccine according to the current invention is ANGPTL3 based vaccine. More preferably, vaccine according to the current invention comprises an antigenic ANGPTL3 peptide.

In one of the embodiments, the present invention provides vaccine composition comprising an antigenic ANGPTL3 peptide optionally linked to an immunogenic carrier. The preferred antigenic ANGPTL3 peptide according to the present invention is the peptide(s) of general formula (I).

In another embodiment, the present invention provides vaccine composition comprising an antigenic ANGPTL3 peptide optionally conjugated with an immunogenic carrier and one or more adjuvants, preferably one or two adjuvants. The preferred antigenic ANGPTL3 peptide according to the present invention is the peptide(s) of general formula (I).

In a preferred embodiment, the present invention provides use of vaccine composition for the manufacture of the medicament. Vaccine composition according to the present invention comprising antigenic ANGPTL3 peptide optionally linked to an immunogenic carrier and one or more adjuvants, preferably one or two adjuvants. The preferred antigenic ANGPTL3 peptide according to the present invention is the peptide(s) of general formula (I).

In a preferred embodiment, the adjuvant according to the current invention is selected from alum, alum in combination with MF-59, TLR3 agonist selected from Poly(I.C), TLR 4 agonist selected from Monophosphoryl Lipid A or GLA and the like, TLR5 agonist selected from Flagellin, TLR7 agonist selected from Gardiquimod and Imiquimod, TLR7/8 agonist selected from R848, NOD2 agonist selected from N-glycolyl-MDP.CpG-containing nucleic acid (where the cytosine is unmethylated), QS21 (saponin adjuvant), interleukins, beta-sitosterol and the like.

In a further preferred embodiment, the adjuvant according to the current invention is selected from alum, alum in combination with other adjuvants like MF-59, GLA, Monophosphoryl Lipid A, CpG-containing nucleic acid (where the cytosine is unmethylated), QS21 (saponin adjuvant), interleukins, beta-sitosterol.

In another embodiment, the present invention provides use of vaccine composition prepared according to the present invention for the treatment of ANGPTL3-related health disorders. In a preferred embodiment, the present invention provides use of a vaccine composition prepared according to the present invention for the treatment of liver diseases, hyperlipidaemia, hypercholesterolemia, or atherosclerosis including the complications lead to the cardiovascular diseases (CVD) which causes morbidity and mortality wherein effect of ANGPTL3 is detrimental. In a more preferred embodiment, the present invention provides use of a vaccine composition prepared according to the present invention for the treatment of liver disease wherein liver disease is selected from non-alcoholic steatohepatitis (NASH) and non-alcoholic fatty liver disease (NAFLD).

### DETAILED DESCRIPTION OF THE INVENTION

The present invention provides novel approach of a vaccine composition for the treatment of ANGPTL3-related health disorders. ANGPTL3-related health disorders according to the present invention are disorders where expression of ANGPTL3 is required to be controlled to prevent or to treat such disorders or diseases. More preferably, the vaccine of the present invention can be used for the treatment of liver diseases, hyperlipidaemia, hypercholesterolemia, or atherosclerosis including the complications lead to the cardiovascular diseases (CVD) which causes morbidity and mortality, preferably liver diseases are non-alcoholic steatohepatitis (NASH) and non-alcoholic fatty liver disease (NAFLD). Vaccine according to the present invention comprises an antigenic ANGPTL3 peptide which is a modified/truncated ANGPTL3 or modified by replacing the natural amino acid by unnatural amino acids optionally conjugated with suitable immunogenic carrier. An ANGPTL3 peptide according to the present invention acts as an immunogen which is capable of inducing the formation of antibodies directed to angiopoietin-like 3 which blocks the one or all actions of ANGPTL3. In one of the embodiments, an antigenic ANGPTL3 peptide according to the present invention is a portion of ANGPTL3 protein which participates in the interaction of ANGPTL3 with the lipoprotein lipase (LPL), comprising amino acids between 2 and 50 and, when administered to an animal is able to generate the antibodies against ANGPTL3 and lower triglycerides and LDL-C in blood of said subject. The antigenic ANGPTL3 peptide according to the present invention comprises at least 2 amino acids, at least 3 amino acids, at least 4 amino acids, at least 5 amino acids, at least 6 amino acids, at least 7 amino acids, at least 8 amino acids, at least 9 amino acids, at least 10 amino acids, at least 11 amino acids, at least amino acids, at least 13 amino acids, at least 14 amino acids, at least 15 amino acids, at least 16 amino acids, at least 17 amino acids, at least 18 amino acids, at least 19 amino acids, at least 20 amino acids, at least 21 amino acids, at least 22 amino acids, at least 23 amino acids, at least 24 amino acids, at least 25 amino acids, at least 26 amino acids, at least 27 amino acids, at least 28 amino acids, at least 29 amino acids, at least 30 amino acids, at least 31 amino acids, at least 32 amino acids, at least 33 amino acids, at least 34 amino acids, at least 35 amino acids, at least 36 amino acids, at least 37 amino acids, at least 38 amino acids, at least 39 amino acids, at least 40 amino acids, at least 41 amino acids, at least 42 amino acids, at least 43 amino acids, at least 44 amino acids, at least 45 amino acids, at least 46 amino acids, at least 47 amino acids, at least 48 amino acids, at least 49 amino acids or at least 50 amino acids. Further, the antigenic ANGPTL3 peptide according to the present invention comprises amino acids between 2 to 5 amino acid residues, 2 to 7 amino acid residues, 2 to 10 amino acid residues, 2 to 12 amino acid residues, 2 to 15 amino acid residues, 2 to 18 amino acid residues, 2 to 20 amino acid residues, 2 to 22 amino acid residues, 2 to 25 amino acid residues, 2 to 30 amino acid residues, 2 to 33 amino acid residues, 2 to 35 amino acid residues, 2 to 40 amino acid residues, 2 to 42 amino acid residues, 2 to 45 amino acid residues or 2 to 50 amino acid residues. In such embodiment, the portion of ANGPTL3 protein which is used for the development of a vaccine of the present invention is specific epitope 1 (SE1) which is Gln²⁹-His⁵³ or its fragments thereof. Said SE1 domain is present in ANGPTL3 and ANGPTL4 protein. (11) In one of the embodiments, an antigenic ANGPTL3 peptide used for the vaccine preparation according to the present invention has 30-70 amino acid residues of hANGPTL3. An amino acid sequence of hANGPTL3 is available in the art for the skilled person as GenBank #NP_055310. In one of the embodiments, an antigenic ANGPTL3 peptide according to the present invention used for the vaccine development is selected from RFAMLDDVKILANGLLQLGHGLKDFVHKTKGQI or its fragment, EPKSRFAMLDDVKILANGLLQLGHGL or its fragment, and SLSPEPKSRFAMLDDVKILANGLLQLGHGLKDFVHKTKGQIND or its fragment.

In one embodiment, the antigenic peptide according to the present invention is a peptide having amino acid sequence which has amino acid residues within residues 17 to 200, 17 to 100, 17 to 70, 17 to 65, 17 to 60, 17 to 57 or 17 to 50 of hANGPTL3 or its modifications such as insertion, deletion or substitution of at least one amino acid residues from the said sequences. In another embodiment, the antigenic peptides according to the present invention is a peptide having amino acid sequence which has amino acid residues within residues 40 to 200, 40 to 100, 40 to 70, 50 to 200, 50 to 100, 50 to 70, 58 to 200, 58 to 100, 58 to 70, 58 to 68 or 61 to 66 (known as a “heparin-binding motif”) of hANGPTL3.

Above mentioned peptides can be modified according to the present invention wherein modification can be insertion, deletion or substitution of at least one amino acid residues. Modification according to the present invention is a substitution of any of the amino acids with functionally similar amino acids or a substitution of any of the amino acids with D-amino acids or substitution of any of the amino acids with non-natural amino acid. For example, basic amino acids in the selected ANGPTL3 peptide sequence, comprising a positively charged amino group can be replaced with Histidine, Lysine, Omithine and Arginine residues. Aromatic amino acid residues in the selected ANGPTL3 peptide sequence can be replaced with 2-Amino-5-phenyl-pentanoic acid (APPA) or α-methylated APPA (α-MeAPPA) or N-methylated APPA, phenylalanine (Phe), α-methylated phenylalanine (α-MePhe), N-methylated phenylalanine or α-methyl-2-fluorophenylalanine (α-Me-2F-Phe) or α-methyl-2,6-difluorophenylalanine (α-Me-2,6F-Phe) or 2-fluorophenylalanine (−2F-Phe) and their derivatives. Uncharged amino acid residues in the selected ANGPTL3 peptide sequence can be replaced with isoleucine, leucine, alanine, valine, glycine, α-aminobutyric acid (Aib) and their derivatives Acidic or negatively charged amino acids residues in the selected ANGPTL3 peptide sequence can be replaced with glutamic acid, aspartic acid and their derivatives. Some of the representative peptide sequence containing naturally or unnaturally occurring amino acid residues are EPKSRF-Aib-MLDDVKILANGLLQLGHGL KDFVHKTKGQI; EPKSRFAMLDDVKIL-Aib-NGLLQLGHGLKDFVHKTK GQI; RF-Aib-MLDDVKIL-Aib-NGLLQLGHGLKDFVHKTKGQI; EPKS—R(NO₂)—F-Aib-MLDDVKILANGLLQLGHGLKDFVHKTKGQI; EPKS—R(NO₂)—F-AC₆C-MLDDVKILANGLLQLGHGLKDFVHKTKGQI; RF-ACSC-ML-(αMe-Asp)-DVKILANGLLQLGHGLKDFVHKTKGQI; RF-Aib-MLDDVKILANG LL QLGHGLKDFVHKTK-βAla-QI; EPKS—R(NO₂)—F-AC6C-MLDDVKILAN-β Ala-LLQLGHGLKD-(αMe-2,6-diF-Phe)-VHKTKGQI; RF-Aib-MLDDVKILAN-βAla-LLQLGHGLKDFVHK-Thr(OMe)-KGQI. Some modifications according to the current invention includes truncated version of the mentioned amino acid sequences which is capable to elicit the immune response in a stable manner.

Preferably, the present invention relates to novel peptides of general formula (I), which are optionally conjugated with suitable immunogenic carrier. Such peptide(s) can be used as a vaccine or a vaccine composition. In a preferred embodiment, the peptide according to the present invention comprises of 33 amino acid residues of general formula (I),

A-Z₁-Z₂-Z₃-Z₄-Z₅-Z₆-Z₇-Z₈-Z₉-Z₁₀-Z₁₁-Z₁₂-Z₁₃-Z₁₄-Z₁₅-Z₁₆-Z₁₇-Z₁₈-Z₁₉-Z₂₀-Z₂₁-Z₂₂-Z₂₃-Z₂₄-Z₂₅-Z₂₆-Z₂₇-Z₂₈-Z₂₉-Z₃₀-Z₃₁-Z₃₂-Z₃₃—B Formula (I)

Wherein,

‘A’ represents the groups —NH—R₁, R₂—CO—NH—, or —CONHR₁ wherein ‘R₁’ at each occurrence independently represents hydrogen or optionally substituted linear or branched (C₁₋₁₈) alkyl chain; ‘R₂’ is selected from optionally substituted linear or branched (C₁₋₁₈) alkyl chain, (C₁₋₆) alkoxy, (C₃-C₆) cycloalkyl, aryl, heteroaryl or arylalkyl groups;

In one of the embodiments, ‘A’ represents suitable amino acid selected from cysteine, valine, gultamic acid, proline, lysine, serine, leucine, alpha-methyl-valine, Lys(Biotin), Lys(alkyl), Lys(acetyl) and combination thereof. These amino acids are either single or group of amino acids containing up to eight amino acids;

In a preferred embodiment, the aryl group is selected from phenyl, naphthyl, indanyl, fluorenyl or biphenyl, groups and the heteroaryl group is selected from pyridyl, thienyl, furyl, imidazolyl, benzofuranyl groups;

In a preferred embodiment, ‘A’ represents Cysteine or Ser-Leu-Ser-Pro-Glu-Pro-Lys-Ser- or its suitable derivatives and Glu-Pro-Lys-Ser- or its suitable derivatives. Derivatives as referred herein may include derivative with non-natural amino acids.

‘B’ represents R₃, —COOR₃, —CONHR₃, CH₂OR₃ or —NH—R₃ wherein R₃ at each occurrence independently represents H or suitable amino acid selected from serine, cysteine, valine, asparagine, glutamic acid, aspartic acid, alpha-methyl-valine, Lys(Biotin), Lys(alkyl), Lys(acetyl) and these amino acids are either single or group of amino acids containing up to two amino acids; Each of Z₁, Z₉, Z₁₃, Z₁₇, Z₂₃, Z₂₈, Z₃₀, Z₃₂ independently represents an amino acid residue selected from the group of amino acid residues, preferably selected from the group of arginine, glutamine, lysine, asparagine, homoarginine, citruline, ornithine, histidine, 2-amino-4-cyanobutanoic acid (Abu(CN)) and their suitable derivatives;

Each of Z₂ and Z₂₅ independently represents amino acid residues selected from the group of phenylalanine, tyrosine, tryptophan and their suitable derivatives. Derivatives of Z₂ and Z₂₅ may be independently selected from 2-fluorophenylalanine, 2-aminophenyl pentanoic acid, alpha-methyl-2-aminophenyl pentanoic acid, alpha-methyl-phenylalanine, alpha-methyl-2-fluorophenylalanine, alpha-methyl-2, 6-diflurophenyl alanine, 2-Pyridylalanine, 3-Pyridylalanine, 4-Pyridylalanine, (2-Thienyl)-alanine and (4-Thiazolyl)-alanine; Each of Z₃, Z₈, Z₁₂, Z₁₄, Z₁₉, Z₂₁, Z₂₆, Z₃₁ independently represents amino acid residues selected from the group of uncharged amino acid residues, preferably selected from the group of glycine, alanine, serine, threonine, valine and suitable their derivatives. Derivatives of Z₃, Z₈, Z₁₂, Z₁₄, Z₁₉, Z₂₁, Z₂₆, and Z₃₁ may be independently selected from Aib, (AC₃C—OH), (AC₅C—OH), (AC₆C—OH), sarcosine, N-methyl-alanine, beta alanine and the like;

Z₄ represents a naturally or unnaturally occurring amino acid selected from the group comprising of Met, N-methyl-Met ((NMe) M), alpha-methyl-Met (αMe-M), Ethionine (EtMet), selenomethionine (SMet);

Each of Z₅, Z₁₀, Z₁₁, Z₁₅, Z₁₆, Z₁₈, Z₂₂ and Z₃₃ independently represents amino acid residues selected from the group of uncharged amino acid residues, preferably selected from the group of isoleucine, leucine, norleucine, glycine, alanine, beta alanine (βAla), Aib and their suitable derivatives. Derivatives of Z₅, Z₁₀, Z₁₁, Z₁₅, Z₁₆, Z₁₈, Z₂₂ and Z₃₃ may be independently selected from N-methyl-isoleucine, N-methyl-leucine, Nva, HoLeu and alpha-methyl-leucine and the like;

Each of Z₆, Z₇, Z₂₄, independently represents amino acid residues selected from the group of hydrophilic, negatively charged amino acid residues, preferably an amino acid residue selected from the group comprising of glutamic acid, aspartic acid and their derivatives. Derivatives of Z₆, Z₇ and Z₂₄ may be independently selected from alpha-methyl-aspartic acid, alpha-methyl-glutamic acid and homoglutamic acid and the like;

Each of Z₂₀ and Z₂₇ independently represents a naturally or unnaturally occurring amino acid selected from the group comprising of histidine, glutamine, asparagine and their suitable derivatives;

Z₂₉ represents an amino acid residue selected from the group comprising of uncharged amino acid residues, preferably selected from the group comprising of threonine, serine, valine, alanine and their suitable derivatives. Derivative of Z₂₉ is selected from homoserine, O-methyl-threonine, O-methyl-serine and O-methyl-homoserine and the like;

with the proviso that the formula (I) does not include the peptide of SEQ ID NO. 1.

In one of the preferred embodiments, a series of ANGPTL3 peptides according to the present invention is as described herein with general formula (I):

A-Z₁-Z₂-Z₃-Z₄-Z₅-Z₆-Z₇-Z₈-Z₉-Z₁₀-Z₁₁-Z₁₂-Z₁₃-Z₁₄-Z₁₅-Z₁₆-Z₁₇-Z₁₈-Z₁₉-Z₂₀-Z₂₁-Z₂₃-Z₂₄-Z₂₅-Z₂₆-Z₂₇-Z₂₈-Z₂₉-Z₃₀-Z₃₁-Z₃₂-Z₃₃—B Formula (I)

Wherein each of Z₁-Z₃₃ when present, independently represents the naturally occurring amino acid or unnatural/modified amino acids sequences, with the proviso that either one or multiple amino acids of Z₁-Z₃₃ independently represents an unnatural/modified amino acid. In one of the embodiments, all the amino acids in Z₁-Z₃₃ peptide sequence may be present or some amino acids may have deleted or absent, which may be either single amino acid or multiple amino acids, preferably up to 7 amino acid may be deleted or absent. Essentially, the said series of ANGPTL3 peptides according to the present invention is as described herein with general formula (I) does not include the peptide of SEQ ID NO. 1.

In one of the preferred embodiments, the peptide is selected from the group comprising of SEQ ID Nos. 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 and 111.

In one of the embodiments, the peptide sequences encompassing conservative and/or functionally similar substitutions of amino acids in the peptide(s) of the present invention are also within the scope of the invention, provided that the biological activity is retained. Functionally similar amino acids can be classified in neutral-weakly hydrophobic amino acids (Ala, Gly, Pro, Ser, Thr), hydrophilic-amine group containing amino acids (Asn, Gln), hydrophilic-acidic amino acids (Asp, Glu) hydrophilic-basic amino acids (Arg, His, Lys), hydrophobic amino acids (Ile, Met, Leu, Val) and hydrophobic-aromatic amino acids (Phe, Trp, Tyr). In accordance with the present invention, the various novel peptides having the structural formula (I) were synthesised and then conjugated with a suitable immunogenic carrier. These vaccines can be useful in the treatment or prevention of diseases mediating through ANGPTL3.

Preparation of the Peptides

Several synthetic routes can be employed to prepare the peptides of the present invention well known to one skilled in the art of peptide synthesis. The peptide(s) of general formula (I), where all symbols are as defined herein above can be synthesized using the methods described below, together with conventional techniques known to those skilled in the art of peptide synthesis or variations thereon as appreciated by those skilled in the art. Referred methods include, but not limited to those described below. The peptides thereof described herein may be produced by chemical synthesis using suitable variations of both the solution-phase (preferably, using Boc-chemistry, references 12 and 13) and/or solid-phase techniques, such as those described in references 14, 15 and 16.

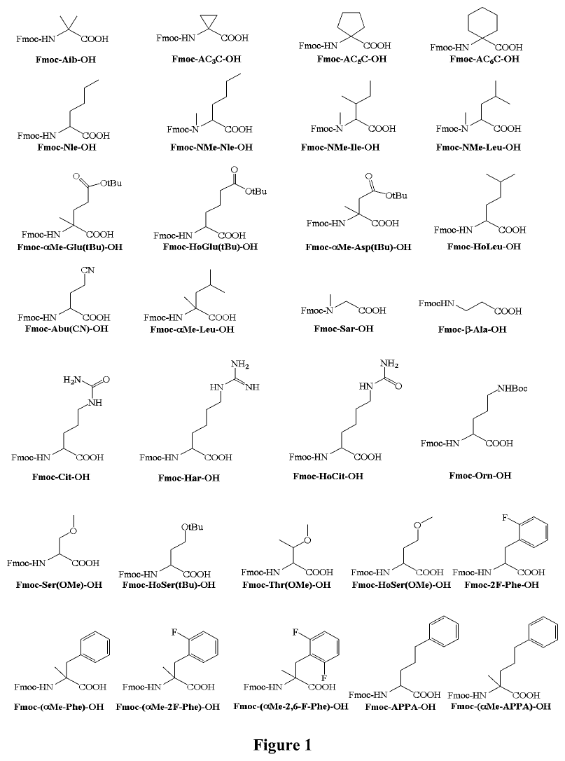

The preferred strategy for preparing the peptides of this invention is based on the use of Fmoc-based SPPS approach, wherein Fmoc (9-fluorenylmethoxycarbonyl) group is used for temporary protection of the α-amino group in combination with the acid labile protecting groups, such as tert-butoxycarbonyl (Boc), tert-butyl (But), Trityl (Trt) groups (FIG. 1), for temporary protection of the amino acid side chains, if present (see for example references 17 and 18).

The peptides can be synthesized in a stepwise manner on an insoluble polymer support (resin), starting from the C-terminus of the peptide. In an embodiment, the synthesis is initiated by appending the C-terminal amino acid of the peptide to the resin through formation of an amide, ester or ether linkage. This allows the eventual release of the resulting peptide as a C-terminal amide, carboxylic acid or alcohol, respectively.

In the Fmoc-based SPPS, the C-terminal amino acid and all other amino acids used in the synthesis are required to have their α-amino groups and side chain functionalities (if present) differentially protected (orthogonal protection), such that the α-amino protecting group may be selectively removed during the synthesis, using suitable base such as 20% piperidine solution, without any premature cleavage of peptide from resin or deprotection of side chain protecting groups, usually protected with the acid labile protecting groups.

The coupling of an amino acid is performed by activation of its carboxyl group as an active ester and reaction thereof with unblocked α-amino group of the N-terminal amino acid appended to the resin. After every coupling and deprotection, peptidyl-resin was washed with the excess of solvents, such as DMF, DCM and diethyl ether. The sequence of α-amino group deprotection and coupling is repeated until the desired peptide sequence is assembled (Scheme 1). The peptide is then cleaved from the resin with concomitant deprotection of the side chain functionalities, using an appropriate cleavage mixture, usually in the presence of appropriate scavengers to limit side reactions. The resulting peptide is finally purified by reverse phase HPLC.

The synthesis of the peptidyl-resins required as precursors to the final peptides utilizes commercially available cross-linked polystyrene polymer resins (Novabiochem, San Diego, Calif.). Preferred resin for use in this invention is Fmoc-PAL-PEG-PS resin, 4-(2′, 4′-dimethoxyphenyl-Fmoc-aminomethyl)-phenoxyacetyl-p-methyl benzhydrylamine resin (Fmoc-Rink amide MBHA resin), 2-chloro-Trityl-chloride resin or p-benzyloxybenzyl alcohol resin (HMP resin) to which the C-terminal amino acid may or may not be already attached. If the C-terminal amino acid is not attached, its attachment may be achieved by HOBt active ester of the Fmoc-protected amino acid formed by its reaction with DIPCDI. In case of 2-Chloro-trityl resin, coupling of first Fmoc-protected amino acid was achieved, using DIPEA. For the assembly of next amino acid, N-terminal protection of peptidyl resin was selectively deprotected using 10-20% piperidine solution. After every coupling and deprotection, excess of amino acids and coupling reagents were removed by washing with DMF, DCM and ether. Coupling of the subsequent amino acids can be accomplished using HOBt or HOAt active esters produced from DIPCDI/HOBt or DIPCDI/HOAt, respectively. In case of some difficult coupling, especially coupling of those amino acids, which are hydrophobic or amino acids with bulky side chain protection; complete coupling can be achieved using a combination of highly efficient coupling agents such as HBTU, PyBOP or TBTU, with additives such as DIPEA.

The synthesis of the peptides described herein can be carried out by using batch wise or continuous flow peptide synthesis apparatus, such as CS-Bio or AAPPTEC peptide synthesizer, utilizing the Fmoc/t-butyl protection strategy. Optionally, the non-commercial amino acid(s) that are non-natural present at different position were incorporated into the peptide chain, using one or more methods known in the art. In one approach, Fmoc-protected non-natural amino acid was prepared in solution, using appropriate literature procedures. For example, the Fmoc-protected APPA analogues, described above, were prepared from L-pyroglutamic acid, in good enantiomeric purity, using modified literature procedure (19).

The Fmoc-protected α-methylated amino acids were prepared using asymmetric Strecker synthesis (20, 21). The resulting derivative was then used in the stepwise synthesis of the peptide. Alternatively, the required non-natural amino acid was built on the resin directly using synthetic organic chemistry procedures and a linear peptide chain were prepared.

The peptide-resin precursors for their respective peptides may be cleaved and deprotected using suitable variations of any of the standard cleavage procedures described in the literature (22). A preferred method for use in this invention is the use of TFA cleavage mixture, in the presence of water and TIPS as scavengers. Typically, the peptidyl-resin was incubated in TFA/Water/TIPS (95:2.5:2.5) for 1.5-4 h at room temperature. The cleaved resin is then filtered off and the TFA solution is concentrated or dried under reduced pressure. The resulting crude peptide is either precipitated or washed with Et₂O or is re-dissolved directly into DMF or 50% aqueous acetic acid for purification by preparative HPLC.

Peptides with the desired purity can be obtained by purification using preparative HPLC. The solution of crude peptide is injected into a semi-Prep column (Luna 10μ; C18; 100 A°), dimension 250×50 mm and eluted with a linear gradient of ACN in water, both buffered with 0.1% TFA, using a flow rate of 40 ml/min with effluent monitoring by PDA detector at 220 nm. The structures of the purified peptides can be confirmed by Electrospray Mass Spectroscopy (ES-MS) analysis.

All the peptide prepared were isolated as trifluoro-acetate salt with TFA as a counter ion, after the Prep-HPLC purification. However, some peptides were subjected for desalting, by passing through a suitable ion exchange resin bed, preferably through anion-exchange resin Dowex SBR P(Cl) or an equivalent basic anion-exchange resin. In some cases, TFA counter ions were replaced with acetate ions, by passing through suitable ion-exchange resin, eluted with dilute acetic acid buffer. For the preparation of the hydrochloride salt of peptides, in the last stage of the manufacturing, selected peptides, with the acetate salt was treated with 4 M HCl. The resulting solution was filtered through a membrane filter (0.2 μm) and subsequently lyophilized to yield the white to off-white HCl salt. Following similar techniques and/or such suitable modifications, which are well within the scope of persons skilled in the art, other suitable pharmaceutically acceptable salts of the peptides of the present invention were prepared.

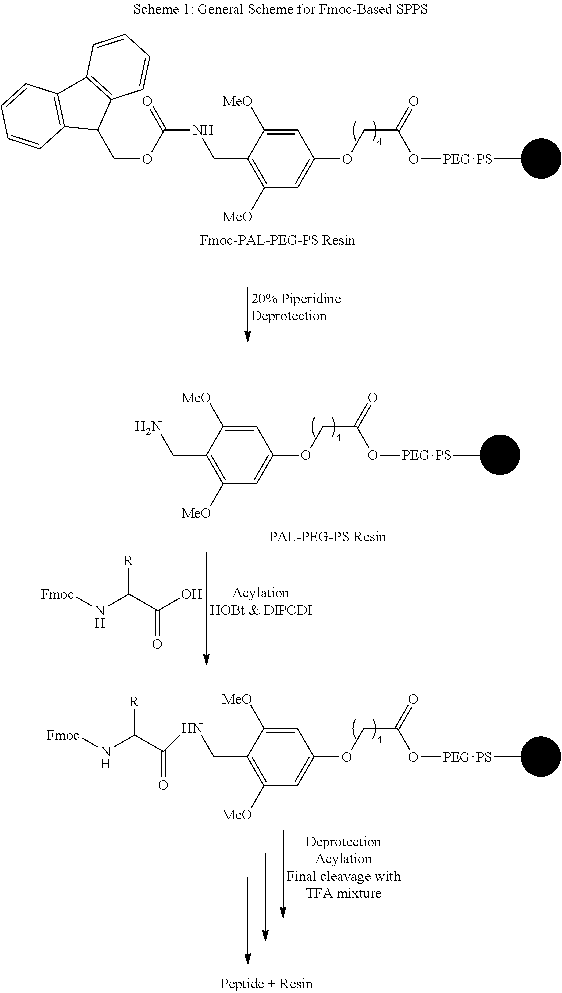

General Method of Preparation of Peptides, Using SPPS Approach:

Assembly of Peptides on Resin:

Sufficient quantity (50-100 mg) of Fmoc-PAL-PEG-PS resin or Fmoc-Rink amide MBHA resin, was loaded: 0.5-0.6 mmol/g was swelled in DMF (1-10 ml/100 mg of resin) for 2-10 minutes. The Fmoc-group on resin was removed by incubation of resin with 10-30% piperidine in DMF (10-30 ml/100 mg of resin), for 10-30 minutes. Deprotected resin was filtered and washed excess of DMF, DCM and ether (50 ml×4). Washed resin was incubated in freshly distilled DMF (1 ml/100 mg of resin), under nitrogen atmosphere for 5 minutes. A 0.5 M solution of first Fmoc-protected amino acid (1-3 eq.), pre-activated with HOBt (1-3 eq.) and DIPCDI (1-2 eq.) in DMF was added to the resin, and the resin was then shaken for 1-3 hrs, under nitrogen atmosphere. Coupling completion was monitored using a qualitative ninhydrin test. After the coupling of first amino acid, the resin was washed with DMF, DCM and Diethyl ether (50 ml×4). For the coupling of next amino acid, firstly, the Fmoc-protection on first amino acid, coupled with resin was deprotected, using a 10-20% piperidine solution, followed by the coupling the Fmoc-protected second amino acid, using a suitable coupling agents, and as described above. The repeated cycles of deprotection, washing, coupling and washing were performed until the desired peptide chain was assembled on resin, as per general (Scheme 1) above. Finally, the Fmoc-protected peptidyl-resin prepared above was deprotected by 20% piperidine treatment as described above and the peptidyl-resins were washed with DMF, DCM and Diethyl ether. Resin containing desired peptide was dried under nitrogen pressure for 10-15 minutes and subjected for cleavage/deprotection.

Using above protocol and suitable variations thereof which are within the scope of a person skilled in the art, the peptides designed in the present invention were prepared, using Fmoc-SPPS approach. Furthermore, resin bound peptides were cleaved and deprotected, purified and characterized using following protocol.

Cleavage and Deprotection:

The desired peptides were cleaved and deprotected from their respective peptidyl-resins by treatment with TFA cleavage mixture as follows. A solution of TFA/Water/Triisopropylsilane (95:2.5:2.5) (10 ml/100 mg of peptidyl-resin) was added to peptidyl-resins and the mixture was kept at room temperature with occasional starring. The resin was filtered, washed with a cleavage mixture and the combined filtrate was evaporated to dryness. Residue obtained was dissolved in 10 ml of water and the aqueous layer was extracted 3 times with ether and finally the aqueous layer was freeze-dried. Crude peptide obtained after freeze-drying was purified by preparative HPLC as follows:

Preparative HPLC Purification of the Crude Peptides

Preparative HPLC was carried out on a Shimadzu LC-8A liquid chromatography.

A solution of crude peptide dissolved in DMF or water was injected into a semi-Prep column (Luna 10μ; C18; 100 Ao), dimension 250×50 mm and eluted with a linear gradient of ACN in water, both buffered with 0.1% TFA, using a flow rate of 15-50 ml/min, with effluent monitoring by PDA detector at 220 nm. A typical gradient of 20% to 70% of water-ACN mixture, buffered with 0.1% TFA was used, over a period of 50 minutes, with 1% gradient change per minute. The desired product eluted were collected in a single 10-20 ml fraction and pure peptides were obtained as amorphous white powders by lyophilisation of respective HPLC fractions.

HPLC Analysis of the Purified Short-Chain Peptides

After purification by preparative HPLC as described above, each peptide was analyzed by analytical RP-HPLC on a Shimadzu LC-10AD analytical HPLC system. For analytical HPLC analysis of peptides, Luna 5μ; C18; 100 A□, dimension 250×4.6 mm column was used, with a linear gradient of 0.1% TFA and ACN buffer and the acquisition of chromatogram was carried out at 220 nm, using a PDA detector.

Characterization by Mass Spectrometry

Each peptide was characterized by electrospray ionization mass spectrometry (ESI-MS), either in flow injection or LC/MS mode. Triple quadrupole mass spectrometers (API-3000 (MDS-SCIES, Canada) was used in all analyses in positive and negative ion electrospray mode. Full scan data was acquired over the mass range of quadrupole, operated at unit resolution. In all cases, the experimentally measured molecular weight was within 0.5 Daltons of the calculated monoisotopic molecular weight. Quantification of the mass chromatogram was done using Analyst 1.4.1 software.

Following table 1 is the list of peptides synthesized using the SPPS approach as described above.

TABLE 1
List of peptides prepared according to the present invention

| Seq. | |
|---|---|
| ID. | |
| No. | Sequence of peptides |
| 1 | RFAMLDDVKILANGLLQLGHGLKDFVHKTKGQI |
| 2 | R-APPA-AMLDDVKILANGLLQLGHGLKDFVHKTKGQI |
| 3 | R-αMePhe-AMLDDVKILANGLLQLGHGLKDFVHKTKGQI |
| 4 | R-Aib-AMLDDVKILANGLLQLGHGLKDFVHKTKGQI |
| 5 | CRFAM-Nle-DDVKILANGLLQLGHGLKDFVHKTKGQI |
| 6 | RFAMLDDV-Cit-ILANGLLQLGHGLKDFVHKTKGQI |
| 7 | RFAMLDDV-Or-ILANGLLQLGHGLKDFVHKTKGQI |
| 8 | RFAMLDDVKILAN-Sar-LLQLGHGLKDFVHKTKGQI |
| 9 | RFAMLDDVKILANGLLQ-αMeLeu-GHGLKDFVHKTKGQI |
| 10 | RFAMLDDVKILANGLLQ-NMeLeu-GHGLKDFVHKTKGQI |
| 11 | RFAMLDDVKILANGLLQL-Sar-HGLKDFVHKTKGQI |
| 12 | RFAMLDDVKILANGLLQLGHGL-Cit-DFVHKTKGQI |
| 13 | RFAMLDDVKILANGLLQLGHGL-Orn-DFVHKTKGQI |
| 14 | RFAMLDDVKILANGLLQLGHGLKD-APPA-VHKTKGQI |
| 15 | RFAMLDDVKILANGLLQLGHGLKD-MePhe-VHKTKGQI |
| 16 | RFAMLDDVKILANGLLQLGHGLKDF-Nva-HKTKGQI |
| 17 | RFAMLDDVKILANGLLQLGHGLKDFVHKT-Cit-GQI |
| 18 | RFAMLDDVKILANGLLQLGHGLKDFV-Cit-KTKGQI |
| 19 | RFAMLDDVKILANGLLQLGHGLKDFVH-Orn-TKGQI |
| 20 | RFAMLDDVKILANGLLQLGHGLKDFVHKT-Orn-GQI |
| 21 | RFAMLDDVKILANGLLQLGHGLKDFVHKTK-Aib-QI |
| 22 | RFAMLDDVKILANGLLQLGHGLKDFVHKTK-Sar-QI |
| 23 | RFAMLDDVKILANGLLQLGHGLKDFVHKTKGQ-αMeIle |
| 24 | RF-Aib-MLDDV-Cit-ILANGLLQLGHGLKDFVHKTKGQI |
| 25 | RFAMLDD-Cit-ILANGLLQLGH-Sar-LKDFVHKTKGQI |
| 26 | RF-Aib-MLDDVKILANGLLQLGH-Sar-LKDFVHKTKGQI |
| 27 | RFAMLDD-Nva-KILANGLLQLGHGL-Cit-DFVHKTKGQI |
| 28 | R-αMePhe-AMLDDVKILANGLLQLGHGL-Orn-DFVHKTKGQI |
| 29 | RFAMLDDVKIL-Aib-NGLLQLGHGLKD-APPA-VHKTKGQI |
| 30 | RFAMLDDVKILANG-Nle-LQLGHGLKD-αMePhe-VHKTKGQI |
| 31 | RFAM-HoLeu-DDVKILANGLLQLGHGLKDF-Nva-HKTKGQI |
| 32 | Har-FAMLDDVKILANGLLQLGHGLKDFVH-Cit-TKGQI |
| 33 | RF-Aib-MLDDVKILANGLLQLGHGLKDFVH-Orn-TKGQI |
| 34 | RFAMLDDVKILANGL-αMeLeu-QLGHGLKDFVHKT-Cit-GQI |
| 35 | Har-FAMLDDVKIL-Aib-NGLLQLGHGLKDFVHKT-Orn-GQI |
| 36 | RF-Aib-MLDDVKILANGLLQLGHG-HoLeu-KDFVHKTK-Aib-QI |
| 37 | RFAMLDDVKIL-Aib-NGL-Holeu-QLGHGLKDFVHKTK-Sar-QI |
| 38 | Har-FAMLDDVKIL-Aib-NGLLQLGH-Sar-LKDFVHKTKGQ-αMeIle |
| 39 | Har-FAMLDDVKILANG-Nle-LQLGHGLKD-aMePhe-VHKTKGQ- |
| | Nle |
| 40 | RFAMLDDVKILANG-HoLeu-LQLGHGLKDF-Nva-HKT-Cit-GQI |
| 41 | Har-FAMLDDVKIL-Aib-NGLLQLGHGL-Cit-DFVHKTKGQI |
| 42 | RF-Aib-MLDDVKIL-Aib-NGL-Nle-QLGHGLKDF-Nva-HKT-Orn- |
| | GQI |
| 43 | Arg(NO₂)-FAMLDDVKILANGL-MeLeu-QLGHGLKDF-Nva-HKT- |
| | Cit-GQI |
| 44 | Arg(NO₂)-FAMLDDVKIL-Aib-NGLLQL-Sar-HGLKDFVH-Cit- |
| | TKGQI |
| 45 | RF-Aib-MLDDVKILAN-Sar-LLQ-HoLeu-GHGLKD-APPA-VHKTK- |
| | Aib-QI |
| 46 | RFMLDDVKIL-Aib-NGL-Holeu-QLGHGLKD-APPA-VHKTK-Sar- |
| | Q-Nle |
| 47 | Har-F-Aib-MLDDVKIL-bAla-NGLLQL-Sar-HGLKDFVHKTKGQ- |
| | αMeIle |
| 48 | RFAMLDDVKILANGLLQLGHGLKDFVHKTKGQIC |
| 49 | CRFAMLDDVKILANGLLQLGHGLKDFVHKTKGQI |
| 50 | RFAMLDDVKIL-Aib-NGLLQLGHGLKDFVHKTKGQIC |
| 51 | CRFAMLDDVKIL-Aib-NGLLQLGHGLKDFVHKTKGQI |
| 52 | RF-AC₃C-MLDDVKILANGLLQLGHGLKDFVHKTKGQI |
| 53 | RFAMLDDVKIL-ACC-NGLLQLGHGLKDFVHKTKGQI |
| 54 | R(NO₂)-F-AC₅C-MLDDVKILANGLLQLGHGLKDFVHKTKGQI |
| 55 | RF-Aib-ML-(αMe-Asp)-DVKILANGLLQLGHGLKDFVHKTKGQI |
| 56 | R-(αMe-2,6-diF-Phe)-AMLDDVKI-HoLeu-ANGLLQLGHGLKD- |
| | (αMe-2,6-diF-Phe)-VHKTKGQI |
| 57 | RF-AC₃C-MLDDVKILAN-βAla-LLQLGHGLKD-(αMe-2,6-diF-Phe)- |
| | VHKTKGQI |
| 58 | RF-Aib-MLDDVKILANGLLQLGHGLKDFVHK-Thr(OMe)-KGQI |
| 59 | RF-Aib-MLDDVKILAN-βAla-LLQLGHGLKDFVHK-Thr(OMe)- |
| | KGQI |
| 60 | EPKSRF-Aib-MLDDVKILANGLLQLGHGLKDFVHKTKGQI |
| 61 | EPKSRFAML DDVKIL-Aib-NGLLQLGHGLKDFVHKTKGQI |
| 62 | EPKS-R(NO₂)-F-Aib- |
| | MLDDVKILANGLLQLGHGLKDFVHKTKGQI |
| 63 | EPKS-R(NO₂)-F-AC₆C -MLDDVKILANGLLQLGHGLKDFVHK |
| | TKGQI |
| 64 | EPKS-R(NO₂)-F-AC₆C-MLDDVKILAN-βAla-LLQLGHGLKD-(aMe- |
| | 2,6-diF-Phe)-VHKTKGQI |
| 65 | SLSPEPKSRFAMLDDVKILANGLLQLGHGLKDFVHKTKGQIND |
| 66 | SLSPEPKSRF-Aib- |
| | MLDDVKILANGLLQLGHGLKDFVHKTKGQIND |
| 67 | SLSPEPKSRFAMLDDVKILANGLLQLGHGL-Cit- |
| | DFVHKTKGQIND |
| 68 | SLSPEPKSRFAMLDDVKILANGLLQLGHGLKD-(αMe-2,6-diF- |
| | Phe)-VHKTKGQIND |
| 69 | SLSPEPKSRFAMLDDVKI-Nle- |
| | ANGLLQLGHGLKDFVHKTKGQIND |
| 70 | EPKSRFAMLDDVKILANGLLQLGHGL |
| 71 | EPKSRF-Aib-MLDDVKILANGLLQLGHGL |
| 72 | EPKSRFAMLDDVKILANGLLQL-Sar-HGL |
| 73 | EPKSRFAMLDDV-Cit-ILANGLLQLGHGL |
| 74 | EPKSRFAMLDD-Nva-KILANGLLQLGHGL |
| 75 | RF-Aib-MLDDVKIL-Aib-NGLLQLGH GLKDFVHKTKGQI |
| 76 | RF-ACC-ML-(αMe-Asp)- |
| | DVKILANGLLQLGHGLKDFVHKTKGQI |
| 77 | RF-Aib-MLDDVKILANGLL QLGHGLKDFVHKTK-βAla-QI |
| 78 | RF-Aib-MLDDVKILANGLLQLGHGLKDF VHKTKGQI |
| 79 | Cit-FAMLDDV-Cit-ILANGLLQLGHGLKDF VHKTKGQI |
| 80 | Har-FAMLDDVKILANGLLQLGHGLKDF VHKTKGQI |
| 81 | Cit-FAMLDDVLILANGLLQLGHGLKDF VHKTKGQI |
| 82 | C-Cit-FAMLDDV-Cit-ILANGLLQLGHGLKDF VHKTKGQI |
| 83 | C-Har-FAMLDDVKILANGLLQLGHGLKDF VHKTKGQI |
| 84 | C-RF-Aib-MLDDVKILANGLLQLGHGLKDF VHKTKGQI |
| 85 | C-Cit-FAMLDDVLILANGLLQLGHGLKDF VHKTKGQI |
| 86 | C-R-APPA-AMLDDVKILANGLLQLGHGLKDFVHKTKGQI |
| 87 | C-RFAMLDDV-Cit-ILANGLLQLGHGLKDFVHKTKGQI |
| 88 | CRFAMLDDV-Cit-IL-Aib-NGLLQLGHGLKDFVHKTKGQI |
| 89 | CRFAM-Nle-DDV-Cit-ILANGLLQLGHGLKDFVHKTKGQI |
| 90 | CRF-Aib-MLDDV-Cit-ILANGLLQLGHGLKDFVHKTKGQI |
| 91 | CR-APPA-AMLDDV-Cit-ILANGLLQLGHGLKDFVHKTKGQI |
| 92 | C-Har-FAMLDDV-Cit-ILANGLLQLGHGLKDFVHKTKGQI |
| 93 | CRF-Aib-MLDDV-Cit-IL-Aib-NGLLQLGHGLKDFVHKTKGQI |
| 94 | C-Har-F-Aib-MLDDV-Cit-IL-Aib-NGLLQLGHGLKDFVHKTKGQI |
| 95 | C-Har-F-Aib-M-Nle-DDV-Cit-IL-Aib- |
| | NGLLQLGHGLKDFVHKTKGQI |
| 96 | RFAMLDDV-Cit-IL-Aib-NGLLQLGHGLKDFVHKTKGQIC |
| 97 | RFAM-Nle-DDV-Cit-ILANGLLQLGHGLKDFVHKTKGQIC |
| 98 | RF-Aib-MLDDV-Cit-ILANGLLQLGHGLKDFVHKTKGQIC |
| 99 | R-APPA-AMLDDV-Cit-ILANGLLQLGHGLKDFVHKTKGQIC |
| 100 | Har-FAMLDDV-Cit-ILANGLLQLGHGLKDFVHKTKGQIC |
| 101 | RF-Aib-MLDDV-Cit-IL-Aib-NGLLQLGHGLKDFVHKTKGQIC |
| 102 | Har-F-Aib-MLDDV-Cit-IL-Aib-NGLLQLGHGLKDFVHKTKGQIC |
| 103 | Har-F-Aib-M-Nle-DDV-Cit-IL-Aib- |
| | NGLLQLGHGLKDFVHKTKGQIC |
| 104 | EPKSRF-Aib-MLDDVKI LANGLLQLGHGLKDFVHKTKGQI |
| 105 | EPKSRFAMLDDVKIL-Aib-NGLLQLGHGLKDFVHKTKGQI |
| 106 | RF-Aib-MLDDVKIL-Aib-NGLLQLGHGLKDFVHKTKGQI |
| 107 | RF-AC₅C-ML-(αMe-Asp)-DVKILANGLLQLGHGLKDFVHKTKGQI |
| 108 | RF-Aib-ML DDVKILANGLLQLGHGLKDFVHKTK-βAla-QI |
| 109 | EPKS-R(NO₂)-F-AC₆C- |
| | MLDDVKILANGLLQLGHGLKDFVHKTKGQI |
| 110 | EPKS-R(NO₂)-F-AC₆C-MLDDVKILAN-βAla-LLQLGHGLKD- |
| | (αMe-2,6-diF-Phe)-VHKTKGQI |
| 111 | RFAM-Nle-DDVKILANGLLQLGHGLKDFVHKTKGQI |

In another preferred embodiment the peptides of the present invention can be chemically synthesized by methods which are well known in the art. It is also possible to produce the peptides of the present invention using recombinant methods. The peptides can be produced in microorganisms such as bacteria such as E. coli, B. subtilis, or any other bacterium that is capable of expressing such peptides, yeast such as Saccharomyces cerevisiae, Schizosaccharomyces pombe, Candida albicans, Pichia pastoris or any other yeast capable of expressing peptides or fungi, in eukaryotic cells such as mammalian or insect cells, or in a recombinant virus vector such as adenovirus, poxvirus, herpes virus, Simliki forest virus, baculovirus, bacteriophage, sindbis virus or Sendai virus. Also methods for isolating and purifying recombinantly produced peptides are well known in the art and include e.g. gel filtration, affinity chromatography, ion exchange chromatography etc.

Conjugation with DT and or Large Protein Molecule

The peptides used in the study were conjugated with Diptheria toxoid (DT) by various methods

Conjugation Using EDAC and ADH

Diptheria toxin is a single polypeptide chain consisting of 535 amino acids, containing two subunits linked by disulfide bridges. One of the subunits binds to the cell surface, allowing the more stable subunit to penetrate the host cell. For conjugation, Diptheria toxoid and peptide taken in equimolar concentration. Concentration of DT and peptide is 2-50 mg/mL. First step is to get the Diptheria toxoid into solution; it was dissolved in Phosphate buffered saline. Next, EDAC (1-ethyl 3, 3 dimethylaminopropyl carbodiimide) was added that provides the first step in cross-linking carboxylic acids. EDAC activates carboxyl groups for direct reaction with primary amines via the amide bond formation, thus primes the Diptheria toxoid for conjugation with peptide. Moreover, EDAC-mediated cross-linking is more effective in acidic pH. Hence, we allowed the reaction to take place by incubating DT with EDAC for one minute in the presence of MES buffer (40-morpholinoethane sulfonic acid) at pH 6.0. Moreover, this EDAC coupling method in the presence of MES improves the efficiency of the conjugation by forming intermediates. Next, ADH (Adipic Acid Dihydrazide) was added, which is a homobifunctional cross-linking reagent that results in relatively stable hydrazone linkages to the DT and peptide. Linking was carried out in a site-specific fashion, by oxidation first and then cross-linking, performed at pH 5.0 (due to the low pKa of the hydrazide). This avoids competition by primary amines. EDAC is again added, and the mixture is incubated for 3 hours at 2-80 C to allow the conjugation to commence. The above prepared DT conjugated peptide is dialyzed through a 10 kD column and sterile filtered (0.2μ filter) for the removal of impurities, and the pure peptide-DT conjugate is stored at 2-80 C for at least a week.

### Method-2: Conjugation Using SMPH (succinimidyl-6-((b-maleimido propion amido) hexanoate)

Activation of DT was done by using desalting columns (Econo-Pac, BioRad, 10 DG). Columns were pre equilibrated with Dulbecco's PBS and allowed the sample to enter the column and first 3.0 ml of effluent was discarded. 4.0 ml of Dulbecco's buffer was added to elute the higher molecular weight component. Eluted sample was collected in glass bottle containing magnetic stirrer so collected the samples with continuous stirring. An approximately 4.0 ml of eluted samples were received. Protein estimation was done in the samples. Considering molecular weight 62 KDa of DT, 50 mM SMPH (succinimidyl-6-((b-maleimidopropionamido) hexanoate) was added slowly in activated DT in dropwise manner with gentle stirring. DT+SMPH mixture was kept on rocking platform for incubation at room temperature for 60 min. Desalting was done in pre equilibrated columns with Dulbecco's PBS as previously and eluted with 4.0 ml of Dulbecco's PBS. Again desalting of each samples was done Protein concentration was measured in samples. From this desalted eluted samples, aliquot of 2.7 ml was kept for reaction with peptide or DMSO (reaction blank). Peptide was dissolved in DMSO. From this peptide solution, 300 μl was added to aliquote of 2.7 ml of eluted sample of DT+SMPH in glass bottle. Further, in reaction blank sample of DT+SMPH, only 300 μl DMSO was added instead of peptide to serve as reaction blank (DT without peptide). Both the samples were incubated on rocking platform for 3 hrs at room temperature. After 3 hrs of incubation for conjugation, desalting was done by Dul-PBS by adding 3.0 ml sample and eluted with 4.0 ml of Dul-PBS. Eluted samples were filtered using syringe filter (0.22 μm). Protein concentration was measured. Confirmation of conjugation using SDS-PAGE was performed in conjugated DT with peptide sample to monitor the gel shift as compared to DT or DT without peptide sample.

Confirmation of Conjugation with DT and or Large Protein Molecule by SDS-PAGE Gel

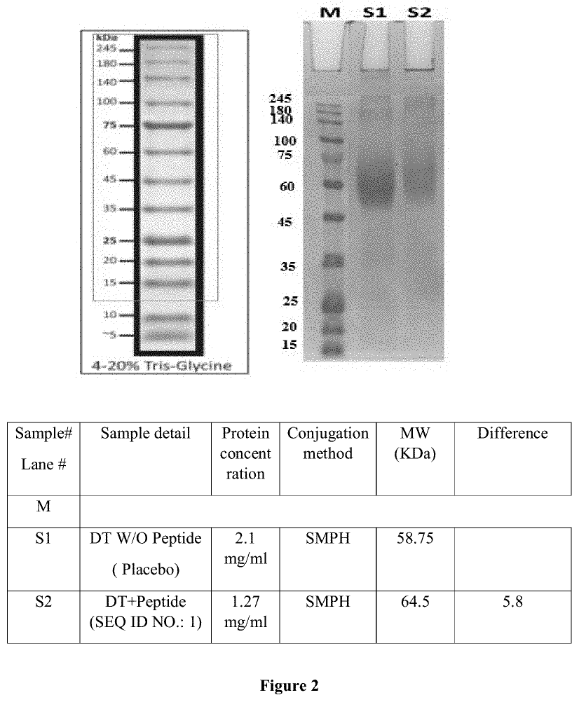

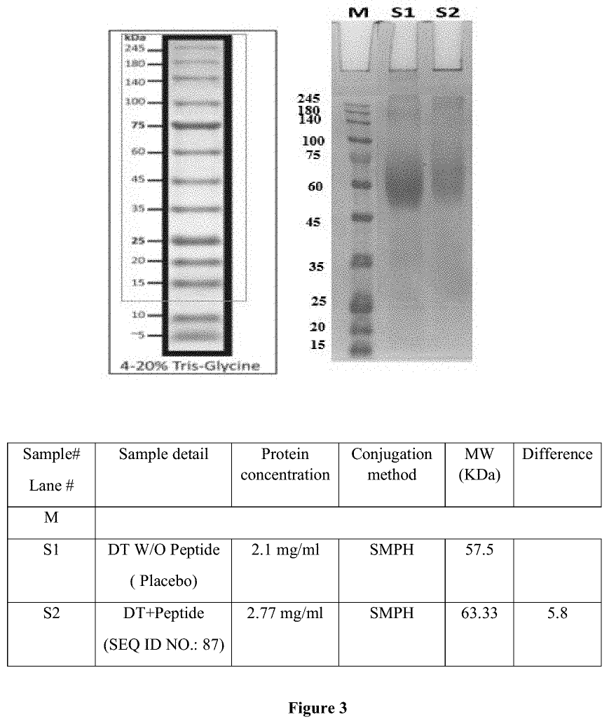

Resolving gel (10%) and stacking gel (5%) were prepared using acrylamide and bisacrylamide for polymerization and cross linking of gel using addition of ammonium persulfate and tetramethylethylenediamine (TEMED). Samples were prepared by addition of loading dye buffer containing bromophenol blue and PBS in conjugated samples (DT without peptide and DT+ peptide to correspond 40-50 μg protein to be loaded in well. After preparation of samples, they were heated at 95° C. for 3-5 min to denature protein which helps in smooth loading of samples in well. Resolving gel was allowed to solidify after pouring in glass slides. Meanwhile electrophoresis buffer was prepared and made the tank assembly ready. Stacking gel was allowed to solidify after pouring over solidified resolving gel. Marker protein was loaded along with prepared samples in wells using pipette. Electric current was applied to run the gel and the bands were separated as per the molecular weight when voltage was applied. Gel was stained using Coomassie stain solution for 2 hr on moving platform. Stain solution was removed and washed the gel with water. Gel was de-stained in de-staining solution containing methanol, glacial acetic acid and miliQ water. Gel was kept for de-staining on moving platform for overnight. De-stained solution was replaced and captured the picture of gel in Image Lab software. Peak area of each band of marker protein, DT without peptide sample and DT+ peptide sample using Image Lab software was measured and calculated the shift in band by difference in molecular weight of band between DT without peptide and DT+ peptide sample. Conjugation of peptide (SEQ ID NO.: 1 and 87) with diphtheria toxoid was done as described herein above. The corresponding gel diagram is given here as FIG. 2(SEQ ID NO.: 1) and FIG. 3 (SEQ ID NO.: 87).

Amount of conjugated product of Diphtheria Toxoid (DT) with peptide and without peptide (reaction blank/placebo) was calculated based upon total protein value of final product, required dose and total volume of formulation to be injected in particular number of animals.

Required amount of conjugate product was pipetted in clean and dry glass bottle. Alum (aluminium hydroxide) was added in dropwise manner with gentle shaking. This mixture was incubated at 2-8° C. for overnight. Next day morning aliquote of Monophosphoryl Lipid A (MPLA) was allowed it to thaw and added in conjugate-alum mixture with gentle shaking. Volume of formulation was made up the by dropwise addition of PBS with gentle shaking. Formulation was allowed to incubate for 1 hour at 2-8° C. and after that it was ready to inject in animals.

In another embodiment, conjugation of the peptide of the present invention is done with CRM197 which is genetically detoxified form of diphtheria toxin by following the general procedure given in WO 2011027257 and prior art.

Affinity Determination of Novel Vaccine Peptides with ANGPTL3 Antibody

Affinity of novel peptides of the present invention with anti-ANGPTL3 antibody ‘ANG’ was analysed by surface plasmon resonance (SPR), using a Biacore instrument (Biacore T200, GE Healthcare). SPR experiments were performed at ° C. with a BIACORE T200 apparatus (GE Healthcare, Uppsala, Sweden).

Surface Preparation: (Procedure for Antibody immobilization) Series S Sensor Chip CM5 surface was activated by a 7-min injection of EDC-NHS. Anti-ANGPTL3 polyclonal antibody (ABC83, Sigma), diluted to 4.5 μg/mL in 10 mM acetate buffer (pH 4.0), was immobilized on one of the four flow cells of a Series S Sensor Chip CM5 with the aim of 10000 RU (resonance units) using amine-coupling. The surface was blocked with 1M ethanolamine (pH 8.5). One flow cell was immobilized as blank for reference subtraction (without antibody). 1×PBS was used as running buffer.

Binding experiments: 1 mM stock of novel peptides of the present invention were made in 1×PBS and binding studies were conducted by passing over blank as well as ligand (anti-ANGPTL3 pAb) immobilized surface. Each cycle consisted of 120 s analyte (novel peptides of the present invention) injection at a flow rate of 10 μL/min (the association phase), followed by a dissociation phase of 300 s. Purified human recombinant ANGPTL3 (FLAG-tag, 91009-1, bps bioscience) was used as positive control (1 μM) and was analyzed under identical condition. Complete dissociation of ANGPTL3 and anti-ANGPTL3 antibody interaction was achieved by regenerating surface with 10 mM glycine/HCl (pH 2.0) for 40 s at a flow rate of 10 μl/min injection.

Data Analysis: The data was analyzed using the Biacore T200 Evaluation software. All the curves were initially reference-subtracted (from blank immobilized surface) and then subtracted from zero analyte concentration. Baselines were adjusted to zero for all curves and data was presented as Relative response or binding RU (Average of 5 s window) 5 s before end of sample injection.

Results:

Affinity Determination of Peptides with Anti-ANGPTL3 Antibody

Affinity of novel peptides of vaccine with ANGPTL3 antibody ANG were analysed by surface plasmon resonance (SPR), using a Biacore instrument (Biacore T200, GE Healthcare). SPR experiments were performed at 25° C. with a BIACORE T200 apparatus (GE Healthcare, Uppsala, Sweden). Results are provided in table 2.

TABLE 2
Results of affinity analysis of anti-PD-1
antibodies by Surface Plasmon Resonance

| SEQ ID No. | Concentration | Relative response |
|---|---|---|
| 1 | 1 mM | 4832.6 |
| 78 | 1 mM | 5539.6 |
| 6 | 1 mM | 6084.1 |
| 79 | 1 mM | 6152.5 |
| 80 | 1 mM | 5308.2 |
| 2 | 1 mM | 5674 |
| 87 | 1 mM | 5820.9 |
| 82 | 1 mM | 5518.3 |
| 83 | 1 mM | 5895.2 |
| 5 | 1 mM | 5850 |
| 86 | 1 mM | 5130.1 |
| 84 | 1 mM | 5813.7 |
| 85 | 1 mM | 4924.7 |
| 49 | 1 mM | 5464.1 |

In a further preferred embodiment, the vaccine prepared according to the present invention may be administered subcutaneously, intramuscularly, intradermally, intravenously (23). The vaccine formulation or vaccine composition may consist of respective carriers, adjuvants, and/or excipients depending on the route of administration.

Further, the present invention provides a vaccine comprising an antigenic ANGPTL3 peptide and optionally an immunogenic carrier. The invention also provides methods for producing such antigenic ANGPLT3 peptide optionally linked to an immunogenic carrier. Preferably, the antigenic ANGPTL3 peptide is linked to an immunogenic carrier.

Immunogenic Carriers of the Invention

In one of the embodiments, the antigenic ANGPTL3 peptide of the invention is linked to an immunogenic carrier molecule to form vaccine, preferably wherein the carrier molecule is not related to the native ANGPTL3 molecule.

The types of carriers used in vaccine composition of the present invention are readily known to the person skilled in the art. Examples of such immunogenic carriers are: serum albumins such as bovine serum albumin (BSA); globulins; thyroglobulins; hemoglobins; hemocyanins (particularly Keyhole Limpet Hemocyanin [KLH]); polylysin; polyglutamic acid; lysine-glutamic acid copolymers; copolymers containing lysine or ornithine; liposome carriers; the purified protein derivative of tuberculin (PPD); inactivated bacterial toxins or toxoids such as tetanus or diptheria toxoid (TT and DT) or fragment C of TT, CRM197 (a nontoxic but antigenically identical variant of diphtheria toxin) other DT point mutants, such as CRM 176, CRM228, CRM 45, CRM 9, CRM 45, CRM 102, CRM 103, CRM 107 and protein D or any other protein or peptide containing helper T-cell epitopes.

In one of the preferred embodiments, the immunogenic carrier according to the present invention is diptheria toxoid (DT).

In another embodiment, the immunogenic carrier according to the present invention is a virus-like particle (VLPs), preferably a recombinant virus-like particle.

The VLP to be used as an immunogenic carrier of the invention is not limited to any specific form. The particle can be synthesized chemically or through a biological process, which can be natural or non-natural. By way of example, this type of embodiment includes a virus-like particle or a recombinant form thereof. In a more specific embodiment, the VLP can comprise, or alternatively consist of, recombinant polypeptides of any of the virus known to form a VLP. The virus-like particle can further comprise, or alternatively consist of, one or more fragments of such polypeptides, as well as variants of such polypeptides. Variants of polypeptides can share, for example, at least 80%, 85%, 90%, 95%, 97%, or 99% identity at the amino acid level with their wild-type counterparts. Variant VLPs suitable for use in the present invention can be derived from any organism so long as they are able to form a “virus-like particle” and can be used as an “immunogenic carrier” as defined herein.

Preferred VLPs according to the present invention include the capsid protein or surface antigen of HBV (HBcAg and HBsAg respectively) or recombinant proteins or fragments thereof, and the coat proteins of RNA-phages or recombinant proteins or fragments thereof, PP7, PPV and Norwalk Virus VLP, more preferably the coat protein of Qbeta or recombinant proteins or fragments thereof.

The antigenic ANGPTL3 peptides of the invention may be coupled to immunogenic carriers via chemical conjugation or by expression of genetically engineered fusion partners. The coupling does not necessarily need to be direct, but can occur through linker sequences. More generally, in the case that antigenic peptides either fused, conjugated or otherwise attached to an immunogenic carrier, spacer or linker sequences are typically added at one or both ends of the antigenic peptides. Such linker sequences generally comprise sequences recognized by the proteasome, proteases of the endosomes or other vesicular compartment of the cell.

In one embodiment, the antigenic ANGPTL3 peptides of the present invention are expressed as fusion proteins with the immunogenic carrier. Fusion of the peptide can be effected by insertion into the immunogenic carrier primary sequence, or by fusion to either the N- or C-terminus of the immunogenic carrier. According to the present invention, fusion proteins of a peptide to an immunogenic carrier is the fusion to either ends of the subunit sequence or internal insertion of the peptide within the carrier sequence are encompassed. Fusion, as referred in the present invention, may be effected by insertion of the antigenic peptide into the sequence of carrier, by substitution of part of the sequence of the carrier with the antigenic peptide, or by a combination of deletion, substitution or insertions.

Furthermore, the invention also provides a vaccine compositions comprising an antigenic ANGPTL3 peptide optionally linked to an immunogenic carrier, optionally comprising one or several adjuvants, preferably one or two adjuvants. Preferably, the vaccine composition comprises one or more adjuvants along with the antigenic ANGPTL3 peptide. Such vaccine compositions, particularly when formulated as pharmaceutical compositions, are deemed useful to prevent, treat or alleviate ANGPTL3-related disorders.

In one of the embodiments, the vaccine formulation that is injected into the mice contains adjuvants with immune-potentiating properties that can direct the immune responses to humoral or cell-mediated immunity, depending on the type of adjuvant.

In order to elicit stronger immune response from the DT-conjugated peptide, additional adjuvants added to the formulation.

Adjuvants of the Present Invention

Exemplary adjuvants to enhance effectiveness of the composition include, but are not limited to alum based adjuvants, mineral salt adjuvants, Complete Freund's adjuvant (CFA), Incomplete Freund's adjuvant (IFA), montanide, MF 59 and Adjuvant 65, bacterially derived adjuvants, lipophilic adjuvants, hydrophilic adjuvants, virosomes or their suitable combinations. Mineral salt adjuvants according to the current invention is selected from salts of calcium, iron and zirconium or their suitable combinations. Lipophilic adjuvant according to the current invention is selected from Telormedix, Mono Phosphoryl Lipid A, glucopyranosyl lipid adjuvant and suitable combinations thereof. Virosomes according to the current invention is selected from immunostimulating reconstituted influenza virosomes (IRIVs) and Respiratory Syncytial Virus virosome (RSV).

Preparation of Alum Based Adjuvant(s):
a. Alum (aluminum hydroxide gel, 2% wet gel suspension) The antigen is slowly released from the insoluble salt particles, allowing prolonged and effective stimulation of the immune system (‘depot effect’) (24). In addition to or in contrast to the depot effect, insoluble aluminium salts activate innate immune cells in a manner that ultimately results in a T helper 2 (Th2)-type immune response Alum induces a Th2 response by improving antigenic uptake of antigen by antigen-presenting cells (APCs) (25).
b. Alum with other adjuvants like Poly (I:C) [TLR3 agonist]/MPLA or GLA-SE [TLR 4 agonist]/Flagellin [TLR5 agonist]/Gardiquimod[TLR7 agonist]/Imiquimod[TLR7 agonist]/R848[TLR7/8 agonist]/N-glycolyl-MDP [NOD2 agonist]

In one of the embodiments, the present invention provides a pharmaceutical composition for inducing an immune response against ANGPTL3 comprising vaccine composition according to the present invention with pharmaceutically acceptable carrier or excipient. Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, micro particles, or in a liposomal preparation. Formulations for parenteral administration may be formulated to be immediate and/or modified release. Modified release formulations include delayed-, sustained-, pulsed-, controlled-, targeted and programmed release.

In further embodiment, the present invention provides use of vaccine composition or an antigenic ANGPTL3 peptide for the manufacture of the medicament. Such medicament can be used to treat NASH or NAFLD and other liver diseases and hyperlipidaemia, hypercholesterolemia, or atherosclerosis including the complications lead to the cardiovascular diseases (CVD) which causes morbidity and mortality.

In a preferred embodiment, the present invention provides vaccine directed to ANGPTL3 comprises effective amount of an antigenic ANGPTL3 peptide as disclosed in the current invention which can elicit an immune response against ANGPTL3. These vaccines can be administered in conventional routes and dosages such as “pharmaceutically effective dose” or “therapeutically effective dose”.

The vaccine according to the present invention is able, when administered to a subject, to lower the TG level, HDL level and/or LDL-cholesterol level in blood of said subject by at least 2%, 5%, 10%, 20%, 30% or 50%.

The vaccine or the vaccine composition according to the present invention can be used as a medicament. The vaccine or the vaccine composition according to the present invention can be used for preventing, alleviating or treating an ANGPTL3-related disorder. The vaccine or the vaccine composition according to the present invention can be used for ANGPTL3-related disorder wherein the ANGPTL3-related disorder is elevated TG level or elevated HDL level or elevated PL level or a condition associated with TG level or elevated HDL level or elevated PL level. In one of the embodiments, the vaccine or the vaccine composition according to the present invention can be used for preventing, alleviating or treating ANGPTL3-related disorder selected from liver diseases, hyperlipidaemia, hypercholesterolemia, or atherosclerosis including the complications lead to the cardiovascular diseases. The liver disease according to the present invention is selected from non-alcoholic steatohepatitis and non-alcoholic fatty liver disease.

Biological Studies

The following examples describe the use of vaccine and its composition with one of an antigenic ANGPTL3 peptide prepared as per the present invention.

### Example 1: Immunogenicity Study in hApoB100/hCETP Double-Transgenic Mice (dTg)

Male or female hApoB100/hCETP dTg mice of more than 8 weeks of age are issued from animal house and kept for 2-3 acclimatization. Mice have access to food and water ad libitum and are kept under a 12 hrs light/dark cycle. On Day-0 (pre-treatment) animals bled and serum are harvested for LDL-cholesterol (LDL-C), total cholesterol, HDL-C and triglycerides measurement. Animals are randomized and grouped to various treatments based on their triglycerides (TG) and LDL-C and body weights. On next day of blood collection, animals are immunised with 0.3 ml of vaccine formulations by subcutaneous or intramuscular route. Next booster injection are given on 2 weeks and 4 weeks after first injection and animals are bled for immunogenicity measurement on two weeks after third injection. Serum is separated and serum LDL-C, total cholesterol, HDL-C and triglycerides levels are measured and immunogenicity or antibody confirmation are done using ELISA for anti-ANGPTL3 antibody titer, binding of serum antibodies with human ANGPTL3 using Surface plasmon resonance (SPR) assay.

Serum LDL-C, HDL-C, total cholesterol and triglycerides levels are determined using commercial kits (Randox Laboratories Ltd, UK) on a Daytona, Randox autoanalyzer (Randox Laboratories Ltd, UK).

### Example 2: Protocol for Immunogenicity and Efficacy in Non-Alcoholic Steatohepatitis (NASH) Induced by CDAHFD (Choline Deficient, L-Amino Acid-Defined, High-Fat Diet) in Male C57 Mice

Nonalcoholic fatty liver disease (NAFLD) is a condition defined by excessive fat accumulation in the form of triglycerides (steatosis, lobular inflammation and progressive pericellular fibrosis in liver. Non-alcoholic steatohepatitis (NASH) is a progressive fibrotic disease, the pathogenesis of which has not been fully elucidated. One of the most common models used in NASH research is a nutritional model where NASH is induced by feeding a diet deficient in both methionine and choline. However, the dietary methionine-/choline deficient model in mice can cause severe weight loss and liver atrophy, which are not characteristics of NASH seen in human patients. The CDAHFD (choline-deficient, L-amino acid-defined, high-fat diet) model overcomes these problems to study the development of NASH-induced fibrosis, and this model has been demonstrated to mimic human NASH in both mice and rats by sequentially producing steatohepatitis, liver fibrosis and liver cancer without any loss of body weight mice. C57BL/6J mice fed with CDAHFD (choline-deficient, L-amino acid-defined, high-fat diet) has increase hepatic steatosis due to impaired hepatic VLDL-TG secretive capacity and causes hepatic insulin resistance due to fat accumulation and TNF-α activation in liver. It is reported that, 6-8 weeks CDAHFD feeding develops NASH in C57 mice (Matsumoto et al., 2013, Int. J. Exp. Path. Pages 1-11-17) which was confirmed in our in-house studies also.

To study the effect of immunogenicity and efficacy of vaccine, we use study protocol in which C57 mice are grouped on the basis of body weight and then fed with CDAHFD (choline-deficient, L-amino acid-defined, high-fat diet). On next day animals are immunised with 0.3 ml of vaccine formulations by subcutaneous or intramuscular route. Next booster injection is given on 2 weeks and 4 weeks after first injection and animals are bled for immunogenicity measurement on two weeks after third injection and subsequently every 2 weeks for measuring the immunogenicity (antibody titers) and efficacy parameters till the 16 weeks. During the whole study period animals are maintained on CDAHF-diet.

Blood samples are collected for estimation of non-fasted serum ALT, AST, TG, TC and levels. Animals are sacrificed at the end of study period; liver will be quickly removed, weighed and fixed in 10% formalin for histological analysis or snap frozen in liquid nitrogen for other assays like liver lipids (TG) analysis are done to evaluate the effect on steatosis.

Light microscopic examination of liver tissue are performed using standard hematoxylin and eosin (H&E) staining. Hepatic fibrosis are accessed by masson's trichrome staining method. Liver specific tissue macrophages i.e. kupffer cells are visualized in liver sections stained with the diastase-periodic acid-schiff method. Fat deposition, if any is demonstrated using oil red-O staining technique on 10% formalin fixed tissue. Specimens are scored as per the scoring method described by Kleiner et al (Hepatology 2005; 41:1313-1321) for the NAFLD Activity Score (NAS).

### Example 3: To Study the Effect of Immunogenicity and Efficacy of Vaccine in Intervention Study Protocol in Treatment of NASH

In this study, it is established the NASH by feeding 8 weeks of CDAHFD. Then, animals are randomised on basis of ALT levels and body weights. On next day animals are immunised with 0.3 ml of vaccine formulations by subcutaneous or intramuscular route. Next booster injection is given on 2 weeks and 4 weeks after first injection and animals are bled for immunogenicity measurement on two weeks after third injection and subsequently every 2 weeks for measuring the immunogenicity (antibody titers) and efficacy parameters mentioned in above protocol till the 16 weeks. During the whole study period animals are maintained on CDAHF-diet.

### Example 4: Immunogenicity Study in High Fat Diet Fed C57 Mice

Male or female mice which are maintained on high fat diet for more than 8 weeks are bled as day-0 (pre-treatment levels). Serum is harvested for Triglycerides (TG), LDL-cholesterol (LDL-C) and total cholesterol measurement. Animals are randomized and grouped to various treatments based on their triglycerides (TG) and LDL-C and body weights. On next day of blood collection, animals are immunised with 0.3 ml of vaccine formulations by subcutaneous or intramuscular route. Next booster injection is given on 2 weeks and 4 weeks after first injection and animals are bled for immunogenicity measurement on two weeks after third injection. Serum is separated and serum LDL-C, total cholesterol and triglycerides levels are measured and immunogenicity or antibody confirmation are done using ELISA for anti-ANGPTL3 antibody titer, binding of serum antibodies with human ANGPTL3 using Surface plasmon resonance (SPR) assay.

### Example 5: Immunogenicity Study in Female Balb/c Mice

Female mice having age 8-12 weeks are bled as day-0 (pre-treatment levels). Serum is harvested for total cholesterol measurement. Animals are randomized and grouped to various treatments based on total cholesterol and body weights. On next day of blood collection animals are immunised with vaccine formulations by subcutaneous or intramuscular route. Next booster injection is given on 2 weeks and 4 weeks after first injection and animals are bled for immunogenicity measurement on two weeks after third injection. Serum is separated and serum total cholesterol levels are measured and immunogenicity or antibody confirmation will be done using ELISA for anti-ANGPTL3 antibody titer, binding of serum antibodies with human ANGPTL3 using Surface plasmon resonance (SPR) assay.

### LIST OF NON-PATENT LITERATURE REFERRED IN THE PRESENT APPLICATION

1. Jensen et al., “S447X variant of the lipoprotein lipase gene, lipids, and risk of coronary heart disease in 3 prospective cohort studies”, American Heart Journal. 2009; 157: 384-390
2. Sagoo et al., “Seven Lipoprotein Lipase Gene Polymorphisms, Lipid Fractions, and Coronary Disease: A HuGE Association Review and Meta-Analysis”, Am J Epidemiol. 2008; 168(11): 1233-1246
3. Khera et al., “Association of Rare and Common Variation in the Lipoprotein Lipase Gene With Coronary Artery Disease”, JAMA. 2017; 317(9): 937-946
4. Dewey et al., “Genetic and Pharmacologic Inactivation of ANGPTL3 and Cardiovascular Disease”, N Engl J Med. 2017; 377(3): 211-221
5. Holland et al., “Acute Noncardiovascular Illness in the Cardiac Intensive Care Unit”, Journal of the American College of Cardiology. 2017; Vol. 69, no. 16
6. Graham et al., “Cardiovascular and Metabolic Effects of ANGPTL3 Antisense Oligonucleotides”, N Engl J Med., 2017; 377(3): 222-32.
7. Yilmaz et al., “Serum concentrations of human angiopoietin-like protein 3 in patients with nonalcoholic fatty liver disease: association with insulin resistance”, European Journal of Gastroenterology & Hepatology. 2009; 21(11): 1247-1251
8. Hess et al., “Analysis of circulating angiopoietin-like protein 3 and genetic variants in lipid metabolism and liver health: the DiOGenes study”, Genes & Nutrition. 2018; Volume 13, 7
9. Chorev et al., “A dozen years of retro-inverso peptidomimetics”, Accounts of Chemical Research. 1993; 26(5): 266-273
10. Olson et al., “Concepts and progress in the development of peptide mimetics”, Journal of Medicinal Chemistry. 1993; 36(21): 3039-3049
11. Lee et al., “Identification of a New Functional Domain in Angiopoietin-Like 3 (ANGPTL3) and Angiopoietin-Like 4 (ANGPTL4) Involved in Binding and Inhibition of Lipoprotein Lipase (LPL)”, J Biol Chem. 2009; 284(20): 13735-45
12. M. Bodansky and A. Bodansky, “The Practice of Peptide Synthesis”, Springer-Verlag, Berlin, 1984
13. E. Gross, J. Meinhofer, “The peptide synthesis, analysis, biology”, Academic Press, London, 1979; Vol. 1
14. G. Barany & R. B. Merrifield, “The peptides: Analysis, synthesis, Biology”; Volume 2—“Special methods in peptide synthesis, Part A”, pp. 3-284
15. E. Gross & J. Meienhofer, eds., “The peptides: Analysis, synthesis, Biology”, Academic Press, New York, 1980
16. J. M. Stewart and J. D. Young, “Solid-phase peptide synthesis”, 2nd Ed., Pierce chemical Co., Rockford, Ill., 1984
17. E. Atherton & R. C. Sheppard, “The Fluorenylmethoxycarbonyl amino protecting group”, in “The peptides: Analysis, synthesis, Biology”; Volume 9—“Special methods in peptide synthesis, Part C”, pp. 1-38
18. S. Udenfriend & J. Meienhofer, eds., “The peptides: Analysis, synthesis, Biology”, Academic Press, San Diego, 1987
19. Betsbrugge et al., “New Amino Acids Derived from L-Pyroglutamic Acid: Synthesis of Trans-4-Benzyl-Cis-5-Phenyl-L-Proline, L-α-(2-Benzyl-3-Phenyipropyl)-Glycine and L-α-(3-Phenylpropyl)-Glycine”, Tetrahedron. 1988, 54: 1753-1762
20. Boesten et al., “Asymmetric Strecker Synthesis of α-Amino Acids via a Crystallization-Induced Asymmetric Transformation Using (R)-Phenylglycine Amide as Chiral Auxiliary”, Organic Letters. 2001; 3(8): 1121-1124
21. Cativiela et al., “Stereoselective synthesis of quaternary α-amino acids.” Part 1: Acyclic compounds, Tetrahedron: Asymmetry. 1998; 9: 3517-3599
22. King et al., “A Cleavage Method Which Minimizes Side Reactions Following Fmoc Solid Phase Peptide Synthesis”, Int J Pept Protein Res. 1990; 36(3): 255-66
23. Sarfaraz K. Niazi, “Handbook of Pharmaceutical Manufacturing Formulations” CRC Press Inc, 2004
24. Glenny A T, Pope C G, Waddington H, Wallace U., “Immunological Notes: XVII-XXIV.” J Pathol Bacteriol. 1926; 29:31-40
25. Grun J L, Maurer P H. “Different T helper cell subsets elicited in mice utilizing two different adjuvant vehicles: the role of endogenous inter leukin 1 in proliferative responses.” Cell Immunol. 1989; 121: 134-145.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A peptide of the following general formula (I):
A-Z₁-Z₂-Z₃-Z₄-Z₅-Z₆-Z₇-Z₈-Z₉-Z₁₀-Z₁₁-Z₁₂-Z₁₃-Z₁₄-Z₁₅-Z₁₆-Z₁₇-Z₁₈-Z₁₉-Z₂₀-Z₂₁-Z₂₂-Z₂₃-Z₂₄Z₂₅-Z₂₆-Z₂₇-Z₂₈-Z₂₉-Z₃₀-Z₃₁-Z₃₂-Z₃₃—B Formula (I)
Wherein,
‘A’ represents the groups —NH—R₁, R₂—CO—NH— or —CONHR₁ or suitable amino acid wherein ‘R₁’ at each occurrence independently represents hydrogen or optionally substituted linear or branched (C₁₋₁₈) alkyl chain;
‘R₂’ is selected from optionally substituted linear or branched (C₁₋₁₈) alkyl chain, (C₁₋₆) alkoxy, (C₃-C₆) cycloalkyl, aryl, heteroaryl or arylalkyl groups;
‘B’ represents R₃, —COOR₃, —CONHR₃, CH₂OR₃ or —NH—R₃, wherein R₃ at each occurrence independently represents hydrogen or suitable amino acid;
each of Z₁, Z₉, Z₁₃, Z₁₇, Z₂₃, Z_{28, Z30}, Z₃₂ independently represents an amino acid residues selected from arginine, glutamine, lysine, asparagine, homoarginine, citruline, ornithine, histidine, 2-amino-4-cyanobutanoic acid (Abu(CN)) and their derivatives;
each of Z_{2 &} Z₂₅ independently represents an amino acid residues selected from phenylalanine, tyrosine, tryptophan and their derivatives;
each of Z₃, Z₈, Z₁₂, Z₁₄, Z₁₉, Z₂₁, Z₂₆, Z₃₁ independently represents an amino acid residues selected from the group of uncharged amino acid residues, preferably selected from the group of glycine, alanine, serine, threonine, valine and their derivatives;
Z₄ represents a naturally or unnaturally occurring amino acid selected from the group comprising of Met, N-methyl-Met ((NMe) M), alpha-methyl-Met (αMe-M), Ethionine (EtMet) or selenomethionine (SMet);
each of Z₅, Z₁₀, Z₁₁, Z₁₅, Z₁₆, Z₁₈, Z₂₂, Z₃₃ independently represents an amino acid residues selected from the group of uncharged amino acid residues, preferably selected from the group of isoleucine, leucine, norleucine, glycine, alanine, beta alanine (βAla), Aib and their derivatives;
each of Z₆, Z₇, Z₂₄, independently represents an amino acid residues selected from the group of hydrophilic, negatively charged amino acid residue, preferably an amino acid residue selected from the group comprising of glutamic acid, aspartic acid and their derivatives;
each of Z₂₀ & Z₂₇ independently represents a naturally or unnaturally occurring amino acid selected from the group comprising of histidine, glutamine, asparagine and their derivatives;
Z₂₉ represents an amino acid residue selected from the group comprising of uncharged amino acid residues, preferably selected from the group comprising of threonine, serine, valine, alanine and their derivatives;
with the proviso that the formula (I) does not include the peptide of SEQ ID NO. 1;
wherein the peptide is antigenic ANGPTL3 peptide.

2. The peptide as claimed in claim 1, wherein the aryl group is selected from phenyl, naphthyl, indanyl, fluorenyl or biphenyl groups.

3. The peptide as claimed in claim 1, wherein the heteroaryl group is selected from pyridyl, thienyl, furyl, imidazolyl or benzofuranyl groups.

4. The peptide as claimed in claim 1, wherein ‘A’ represents single or group of amino acid selected from cysteine, valine, gultamic acid, proline, lysine, serine, leucine, alpha-methyl-valine, Lys(Biotin), Lys(alkyl), Lys(acetyl) and combination thereof.

5. The peptide as claimed in claim 4, wherein the group of amino acid is selected from Ser-Leu-Ser-Pro-Glu-Pro-Lys-Ser- or its suitable derivative(s) and Glu-Pro-Lys-Ser- or its suitable derivative(s).

6. The peptide as claimed in claim 5, wherein derivative is non-natural amino acid.

7. The peptide as claimed in claim 1, wherein ‘B’ represents single or group of suitable amino acid selected from serine, cysteine, valine, asparagine, glutamic acid, aspartic acid, alpha-methyl-valine, Lys(Biotin), Lys(alkyl) or Lys(acetyl).

8. The peptide as claimed in claim 1, wherein the derivatives of Z₂ and Z₂₅ are independently selected from 2-fluorophenylalanine, 2-aminophenyl pentanoic acid, alpha-methyl-2-aminophenyl pentanoic acid, alpha-methyl-phenylalanine, alpha-methyl-2-fluorophenylalanine, alpha-methyl-2,6-diflurophenyl alanine, 2-Pyridylalanine, 3-Pyridylalanine, 4-Pyridylalanine, (2-Thienyl)-alanine or (4-Thiazolyl)-alanine.

9. The peptide as claimed in claim 1, wherein the derivatives of Z₃, Z₈, Z₁₂, Z₁₄, Z₁₉, Z₂₁, Z₂₆, and Z₃₁ are independently selected from Aib, (AC₃C—OH), (AC₅C—OH), (AC₆C—OH), sarcosine, N-methyl-alanine or beta alanine.

10. The peptide as claimed in claim 1, wherein the derivatives of Z₅, Z₁₀, Z₁₁, Z₁₅, Z₁₆, Z₁₈, Z₂₂, Z₃₃ are independently selected from N-methyl-isoleucine, N-methyl-leucine, Nva, HoLeu or alpha-methyl-leucine.

11. The peptide as claimed in claim 1, wherein the derivatives of Z₆, Z₇, Z₂₄ are independently selected from alpha-methyl-aspartic acid, alpha-methyl-glutamic acid or homoglutamic acid.

12. The peptide as claimed in claim 1, wherein the derivatives of Z₂₉ is selected from homoserine, O-methyl-threonine, O-methyl-serine or O-methyl-homoserine.

13. The peptide as claimed in claim 1, wherein each of Z₁-Z₃₃ independently represents the naturally occurring amino acid or unnatural/modified amino acids sequences, with the provisio that at least one or multiple amino acids of Z₁-Z₃₃ independently represents an unnatural/modified amino acid.

14. The peptide as claimed in claim 1, wherein either single amino acid or multiple amino acids in Z₁-Z₃₃ peptide sequence is absent.

15. The peptide as claimed in claim 1, wherein the peptide is selected from the group comprising of SEQ ID Nos. 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 and 111.

16. (canceled)

17. The peptide as claimed in claim 1, wherein the antigenic ANGPTL3 peptide is a portion of ANGPTL3 protein which participates in the interaction of ANGPTL3 with the LPL.

18. The peptide as claimed in claim 1, wherein peptide is coupled to a suitable immunogenic carrier via a suitable linker.

19. The peptide as claimed in claim 18, wherein the linker is 6-maleimido caproic acyl N-hydroxysuccinimide ester.

20. The peptide as claimed in claim 18, wherein the immunogenic carrier is selected from serum albumin, thyroglobulin, hemoglobin, hemocyanin, polylysin, polyglutamic acid, lysine-glutamic acid, copolymers, copolymers containing lysine or ornithine, liposome carrier, purified protein derivative of tuberculin, inactivated bacterial toxin or toxoid, protein D, protein or peptide containing helper T-cell epitopes and VLP.

21. The peptide as claimed in claim 20, wherein the bacterial toxoid is selected from tetanus toxoid, diphtheria toxoid, fragment C of TT, CRM197, CRM 176, CRM228, CRM 45, CRM 9, CRM 45, CRM 102, CRM 103 and CRM 107.

22. The peptide as claimed in claim 21, wherein the bacterial toxoid is selected from diphtheria toxoid and CRM197.

23. The peptide as claimed in claim 20, wherein VLP is selected from HBcAg, HBsAg, Qbeta, PP7, PPV and Norwalk Virus VLP.

24. A vaccine comprising at least one antigenic ANGPTL3 peptide, or a fragment thereof optionally linked to an immunogenic carrier.

25. The vaccine as claimed in claim 24, wherein immunogenic carrier is selected from serum albumin, thyroglobulin, hemoglobin, hemocyanin, polylysin, polyglutamic acid, lysine-glutamic acid, copolymers, copolymers containing lysine or ornithine, liposome carrier, purified protein derivative of tuberculin, inactivated bacterial toxin or toxoid, protein D, protein or peptide containing helper T-cell epitopes and VLP.

26. The vaccine as claimed in claim 25, wherein bacterial toxoid is selected from tetanus toxoid, diphtheria toxoid, fragment C of TT, CRM197, CRM 176, CRM228, CRM 45, CRM 9, CRM 45, CRM 102, CRM 103 and CRM 107.

27. The vaccine as claimed in claim 26, wherein bacterial toxoid is selected from diphtheria toxoid and CRM197.

28. The vaccine as claimed in claim 25, wherein VLP is selected from HBcAg, HBsAg, Qbeta, PP7, PPV and Norwalk Virus VLP.

29. The vaccine as claimed in claim 24, wherein the antigenic ANGPTL3 peptide is selected from signal peptide region of ANGPTL3 or its fragments thereof.

30. The vaccine as claimed in claim 24, for use as a medicament.

31. The vaccine as claimed in claim 24, for preventing, alleviating or treating an ANGPTL3-related disorder.

32. The vaccine as claimed in claim 31, wherein said ANGPTL3-related disorder is selected from liver diseases, hyperlipidaemia, hypercholesterolemia, or atherosclerosis including the complications lead to the cardiovascular diseases.

33. The vaccine as claimed in claim 32, wherein said liver disease is selected from non-alcoholic steatohepatitis and non-alcoholic fatty liver disease.

34. A method for preventing, alleviating or treating an ANGPTL3-related disorder in an individual, comprising administering a therapeutically effective amount of the vaccine as claimed in claim 24.

35. A vaccine composition comprising an antigenic ANGPTL3 peptide as claimed in claim 24 and one or more adjuvants, preferably one or two adjuvants.

36. The vaccine composition as claimed in claim 35, wherein said adjuvant is selected from alum based adjuvant(s), mineral salt adjuvant(s), Complete Freund's adjuvant (CFA), Incomplete Freund's adjuvant (IFA), montanide, MF 59 and Adjuvant 65, bacterially derived adjuvant(s), lipophilic adjuvant(s), hydrophilic adjuvant(s), virosomes or their suitable combinations.

37. The vaccine composition as claimed in claim 36, wherein lipophilic adjuvant(s) is selected from Telormedix, Mono Phosphoryl Lipid A, glucopyranosyl lipid adjuvant and suitable combinations thereof.

38. The vaccine composition as claimed claim 35, for use as a medicament.

39. The vaccine composition as claimed in claim 35, for preventing, alleviating or treating an ANGPTL3-related disorder.

40. The vaccine or the vaccine composition as claimed in claim 39, wherein said ANGPTL3-related disorder is selected from liver diseases, hyperlipidaemia, hypercholesterolemia, or atherosclerosis including the complications lead to the cardiovascular diseases.

41. The vaccine or the vaccine composition as claimed in claim 40, wherein said liver disease is selected from non-alcoholic steatohepatitis and non-alcoholic fatty liver disease.

42. A method for preventing, alleviating or treating an ANGPTL3-related disorder in an individual, comprising administering a therapeutically effective amount of the vaccine composition as claimed claim 35.


SEQUENCE LISTING (111 sequences)

SEQ ID NO: 1 - protein, 33 aa, Artificial Sequence
  artificial ANGPTL3 peptide
RFAMLDDVKILANGLLQLGHGLKDFVHKTKGQI

SEQ ID NO: 2 - protein, 33 aa, Artificial Sequence
  artificial ANGPTL3 peptide
  Xaa (2)..(2): Xaa is APPA 2-Aminophenyl pentanoic acid
RXAMLDDVKILANGLLQLGHGLKDFVHKTKGQI

SEQ ID NO: 3 - protein, 33 aa, Artificial Sequence; identical to SEQ ID NO: 2
  artificial ANGPTL3 peptide
  Xaa (2)..(2): X aa is alpha-Me-Phe alpha-methyl- phenylalanine

SEQ ID NO: 4 - protein, 33 aa, Artificial Sequence; identical to SEQ ID NO: 2
  artificial ANGPTL3 peptide
  Xaa (2)..(2): Xaa is Aib alpha-Amino-isobutyric acid

SEQ ID NO: 5 - protein, 34 aa, Artificial Sequence
  artificial ANGPTL3 peptide
  Xaa (6)..(6): Xaa is Nle Norleucine
CRFAMXDDVKILANGLLQLGHGLKDFVHKTKGQI

SEQ ID NO: 6 - protein, 33 aa, Artificial Sequence
  artificial ANGPTL3 peptide
  Xaa (9)..(9): Xaa  is Cit Citrulline
RFAMLDDVXILANGLLQLGHGLKDFVHKTKGQI

SEQ ID NO: 7 - protein, 33 aa, Artificial Sequence; identical to SEQ ID NO: 6
  artificial ANGPTL3 peptide
  Xaa (9)..(9): Xaa is Orn Ornithine

SEQ ID NO: 8 - protein, 33 aa, Artificial Sequence
  artificial ANGPTL3 peptide
  Xaa (14)..(14): Xaa is Sar Sarcosine
RFAMLDDVKILANXLLQLGHGLKDFVHKTKGQI

SEQ ID NO: 9 - protein, 33 aa, Artificial Sequence
  artificial ANGPTL3 peptide
  Xaa (18)..(18): Xaa is alpha-Me-L alpha-methyl-leucine
RFAMLDDVKILANGLLQXGHGLKDFVHKTKGQI

SEQ ID NO: 10 - protein, 33 aa, Artificial Sequence; identical to SEQ ID NO: 9
  artificial ANGPTL3 peptide
  Xaa (18)..(18): Xaa is NMe-Leu N-methyl-leucine

SEQ ID NO: 11 - protein, 33 aa, Artificial Sequence
  artificial ANGPTL3 peptide
  Xaa (19)..(19): Xaa is Sar Sarcosine
RFAMLDDVKILANGLLQLXHGLKDFVHKTKGQI

SEQ ID NO: 12 - protein, 33 aa, Artificial Sequence
  artificial ANGPTL3 peptide
  Xaa (23)..(23): Xaa is Cit Citrulline
RFAMLDDVKILANGLLQLGHGLXDFVHKTKGQI

SEQ ID NO: 13 - protein, 33 aa, Artificial Sequence; identical to SEQ ID NO: 12
  artificial ANGPTL3 peptide
  Xaa (23)..(23): Xaa is Orn Ornithine

SEQ ID NO: 14 - protein, 33 aa, Artificial Sequence
  artificial ANGPTL3 peptide
  Xaa (25)..(25): Xaa is APPA 2-Aminophenyl pentanoic acid
RFAMLDDVKILANGLLQLGHGLKDXVHKTKGQI

SEQ ID NO: 15 - protein, 33 aa, Artificial Sequence; identical to SEQ ID NO: 14
  artificial ANGPTL3 peptide
  Xaa (25)..(25): Xaa is alpha-Me-Phe alpha-methyl-phenylalanine

SEQ ID NO: 16 - protein, 33 aa, Artificial Sequence
  artificial ANGPTL3 peptide
  Xaa (26)..(26): Xaa is Nva Norvaline
RFAMLDDVKILANGLLQLGHGLKDFXHKTKGQI

SEQ ID NO: 17 - protein, 33 aa, Artificial Sequence
  artificial ANGPTL3 peptide
  Xaa (30)..(30): Xaa is Cit Citrulline
RFAMLDDVKILANGLLQLGHGLKDFVHKTXGQI

SEQ ID NO: 18 - protein, 33 aa, Artificial Sequence
  artificial ANGPTL3 peptide
  Xaa (27)..(27): Xaa is Cit Citrulline
RFAMLDDVKILANGLLQLGHGLKDFVXKTKGQI

SEQ ID NO: 19 - protein, 33 aa, Artificial Sequence
  artificial ANGPTL3 peptide
  Xaa (28)..(28): Xaa is Orn Ornithine
RFAMLDDVKILANGLLQLGHGLKDFVHXTKGQI

SEQ ID NO: 20 - protein, 33 aa, Artificial Sequence; identical to SEQ ID NO: 17
  artificial ANGPTL3 peptide
  Xaa (30)..(30): Xaa is Orn Ornithine

SEQ ID NO: 21 - protein, 33 aa, Artificial Sequence
  artificial ANGPTL3 peptide
  Xaa (31)..(31): Xaa is Aib alpha-Amino-isobutyric acid
RFAMLDDVKILANGLLQLGHGLKDFVHKTKXQI

SEQ ID NO: 22 - protein, 33 aa, Artificial Sequence; identical to SEQ ID NO: 21
  artificial ANGPTL3 peptide
  Xaa (31)..(31): Xaa is Sar Sarcosine

SEQ ID NO: 23 - protein, 33 aa, Artificial Sequence
  artificial ANGPTL3 peptide
  Xaa (33)..(33): Xaa is alpha-Me-IL alpha-methyl-lIsoeucine
RFAMLDDVKILANGLLQLGHGLKDFVHKTKGQX

SEQ ID NO: 24 - protein, 33 aa, Artificial Sequence
  artificial ANGPTL3 peptide
  Xaa (3)..(3): Xaa is Aib alpha-Amino-isobutyric acid
  Xaa (9)..(9): Xaa is Cit Citrulline
RFXMLDDVXILANGLLQLGHGLKDFVHKTKGQI

SEQ ID NO: 25 - protein, 32 aa, Artificial Sequence
  artificial ANGPTL3 peptide
  Xaa (8)..(8): Xaa is Cit Citrulline
  Xaa (20)..(20): Xaa is Sar Sarcosine
RFAMLDDXILANGLLQLGHXLKDFVHKTKGQI

SEQ ID NO: 26 - protein, 33 aa, Artificial Sequence
  artificial ANGPTL3 peptide
  Xaa (3)..(3): Xaa is Aib alpha-Amino-isobutyric acid
  Xaa (21)..(21): Xaa is Sar Sarcosine
RFXMLDDVKILANGLLQLGHXLKDFVHKTKGQI

SEQ ID NO: 27 - protein, 33 aa, Artificial Sequence
  artificial ANGPTL3 peptide
  Xaa (8)..(8): Xaa is Nva Norvaline
  Xaa (23)..(23): Xaa is Cit Citrulline
RFAMLDDXKILANGLLQLGHGLXDFVHKTKGQI

SEQ ID NO: 28 - protein, 33 aa, Artificial Sequence
  artificial ANGPTL3 peptide
  Xaa (2)..(2): Xaa is alpha-Me-Phe alpha-methyl-phenylalanine
  Xaa (23)..(23): Xaa is Orn Ornithine
RXAMLDDVKILANGLLQLGHGLXDFVHKTKGQI

SEQ ID NO: 29 - protein, 33 aa, Artificial Sequence
  artificial ANGPTL3 peptide
  Xaa (12)..(12): Xaa is Aib alpha-Amino-isobutyric acid
  Xaa (25)..(25): Xaa is APPA 2-Aminophenyl pentanoic acid
RFAMLDDVKILXNGLLQLGHGLKDXVHKTKGQI

SEQ ID NO: 30 - protein, 33 aa, Artificial Sequence
  artificial ANGPTL3 peptide
  Xaa (15)..(15): Xaa is Nle Norleucine
  Xaa (25)..(25): Xaa is alpha-Me-Phe alpha-methyl-phenylalanine
RFAMLDDVKILANGXLQLGHGLKDXVHKTKGQI

SEQ ID NO: 31 - protein, 33 aa, Artificial Sequence
  artificial ANGPTL3 peptide
  Xaa (5)..(5): Xaa is HoLeu homoleucine
  Xaa (26)..(26): Xaa is Nva Norvaline
RFAMXDDVKILANGLLQLGHGLKDFXHKTKGQI

SEQ ID NO: 32 - protein, 34 aa, Artificial Sequence
  artificial ANGPTL3 peptide
  Xaa (1)..(1): Xaa is Har homoarginine
  Xaa (28)..(28): Xaa is Cit Citrulline
XFAMLDDVKILANGLLQLGHGLKDFVHXTTKGQI

SEQ ID NO: 33 - protein, 33 aa, Artificial Sequence
  artificial ANGPTL3 peptide
  Xaa (3)..(3): Xaa is Aib alpha-Amino-isobutyric acid
  Xaa (28)..(28): Xaa is Orn Ornithine
RFXMLDDVKILANGLLQLGHGLKDFVHXTKGQI

SEQ ID NO: 34 - protein, 33 aa, Artificial Sequence
  artificial ANGPTL3 peptide
  Xaa (16)..(16): Xaa is alpha-Me-L alpha-methyl-leucine
  Xaa (30)..(30): Xaa is Cit Citrulline
RFAMLDDVKILANGLXQLGHGLKDFVHKTXGQI

SEQ ID NO: 35 - protein, 33 aa, Artificial Sequence
  artificial ANGPTL3 peptide
  Xaa (1)..(1): Xaa is Har homoarginine
  Xaa (12)..(12): Xaa is Aib alpha-Amino-isobutyric acid
  Xaa (30)..(30): Xaa is Orn Ornithine
XFAMLDDVKILXNGLLQLGHGLKDFVHKTXGQI

SEQ ID NO: 36 - protein, 33 aa, Artificial Sequence
  artificial ANGPTL3 peptide
  Xaa (3)..(3): Xaa is Aib alpha-Amino-isobutyric acid
  Xaa (22)..(22): Xaa is HoLeu homoleucine
  Xaa (31)..(31): Xaa is Aib alpha-Amino-isobutyric acid
RFXMLDDVKILANGLLQLGHGXKDFVHKTKXQI

SEQ ID NO: 37 - protein, 33 aa, Artificial Sequence
  artificial ANGPTL3 peptide
  Xaa (12)..(12): Xaa is Aib alpha-Amino-isobutyric acid
  Xaa (16)..(16): Xaa is HoLeu homoleucine
  Xaa (31)..(31): Xaa is Sar Sarcosine
RFAMLDDVKILXNGLXQLGHGLKDFVHKTKXQI

SEQ ID NO: 38 - protein, 33 aa, Artificial Sequence
  artificial ANGPTL3 peptide
  Xaa (1)..(1): Xaa is Har homoarginine
  Xaa (12)..(12): Xaa is Aib alpha-Amino-isobutyric acid
  Xaa (21)..(21): Xaa is Sar Sarcosine
  Xaa (33)..(33): Xaa is alpha-Me-IL alpha-methyl-Isoleucine
XFAMLDDVKILXNGLLQLGHXLKDFVHKTKGQX

SEQ ID NO: 39 - protein, 33 aa, Artificial Sequence
  artificial ANGPTL3 peptide
  Xaa (1)..(1): Xaa is Har homoarginine
  Xaa (15)..(15): Xaa is Nle Norleucine
  Xaa (25)..(25): Xaa is alpha-Me-Phe alpha-methyl-phenylalanine
  Xaa (33)..(33): Xaa is Nle Norleucine
XFAMLDDVKILANGXLQLGHGLKDXVHKTKGQX

SEQ ID NO: 40 - protein, 33 aa, Artificial Sequence
  artificial ANGPTL3 peptide
  X (15)..(15): X is HoLeu homoleucine
  X (26)..(26): X is Nva Norvaline
  X (30)..(30): X is Cit Citrulline
RFAMLDDVKILANGXLQLGHGLKDFXHKTXGQI

SEQ ID NO: 41 - protein, 33 aa, Artificial Sequence
  artificial ANGPTL3 peptide
  Xaa (1)..(1): Xaa is Har homoarginine
  Xaa (12)..(12): Xaa is Aib alpha-Amino-isobutyric acid
  Xaa (23)..(23): Xaa is Cit Citrulline
XFAMLDDVKILXNGLLQLGHGLXDFVHKTKGQI

SEQ ID NO: 42 - protein, 33 aa, Artificial Sequence
  artificial ANGPTL3 peptide
  Xaa (3)..(3): Xaa is Aib alpha-Amino-isobutyric acid
  Xaa (12)..(12): Xaa is Aib alpha-Amino-isobutyric acid
  Xaa (16)..(16): Xaa is Nle Norleucine
  Xaa (26)..(26): Xaa is Nva Norvaline
  Xaa (30)..(30): Xaa is Orn Ornithine
RFXMLDDVKILXNGLXQLGHGLKDFXHKTXGQI

SEQ ID NO: 43 - protein, 32 aa, Artificial Sequence
  artificial ANGPTL3 peptide
  Xaa (1)..(1): Xaa is Arg(NO2)  Arginine (NO2)
  Xaa (15)..(15): Xaa is alpha-Me-L alpha-methyl-leucine
  Xaa (25)..(25): Xaa is Nva Norvaline
  Xaa (29)..(29): Xaa is Cit Citrulline
XFAMLDDVKILANGXQLGHGLKDFXHKTXGQI

SEQ ID NO: 44 - protein, 33 aa, Artificial Sequence
  artificial ANGPTL3 peptide
  Xaa (1)..(1): Xaa is Arg(NO2)  Arginine (NO2)
  Xaa (12)..(12): Xaa is Aib alpha-Amino-isobutyric acid
  Xaa (19)..(19): Xaa is Sar Sarcosine
  Xaa (28)..(28): Xaa is Cit Citrulline
XFAMLDDVKILXNGLLQLXHGLKDFVHXTKGQI

SEQ ID NO: 45 - protein, 33 aa, Artificial Sequence
  artificial ANGPTL3 peptide
  Xaa (3)..(3): Xaa is Aib alpha-Amino-isobutyric acid
  Xaa (14)..(14): Xaa is Sar Sarcosine
  Xaa (18)..(19): Xaa is HoLeu homoleucine
  Xaa (25)..(25): Xaa is APPA 2-Aminophenyl pentanoic acid
  Xaa (31)..(31): Xaa is Aib alpha-Amino-isobutyric acid
RFXMLDDVKILANXLLQXGHGLKDXVHKTKXQI

SEQ ID NO: 46 - protein, 32 aa, Artificial Sequence
  artificial ANGPTL3 peptide
  Xaa (11)..(11): Xaa is Aib alpha-Amino-isobutyric acid
  Xaa (15)..(15): Xaa is HoLeu homoleucine
  Xaa (24)..(24): Xaa is APPA 2-Aminophenyl pentanoic acid
  Xaa (30)..(30): Xaa is Sar Sarcosine
  Xaa (32)..(32): Xaa is Nle Norleucine
RFMLDDVKILXNGLXQLGHGLKDXVHKTKXQX

SEQ ID NO: 47 - protein, 33 aa, Artificial Sequence
  artificial ANGPTL3 peptide
  Xaa (1)..(2): Xaa is Har homoarginine
  Xaa (3)..(3): Xaa is Aib alpha-Amino-isobutyric acid
  Xaa (12)..(12): Xaa is beta-Ala beta alanine
  Xaa (19)..(19): Xaa is Sar Sarcosine
  Xaa (33)..(33): Xaa is alpha-Me-IL alpha-methyl-Isoleucine
XFXMLDDVKILXNGLLQLXHGLKDFVHKTKGQX

SEQ ID NO: 48 - protein, 34 aa, Artificial Sequence
  artificial ANGPTL3 peptide
RFAMLDDVKILANGLLQLGHGLKDFVHKTKGQIC

SEQ ID NO: 49 - protein, 34 aa, Artificial Sequence
  artificial ANGPTL3 peptide
CRFAMLDDVKILANGLLQLGHGLKDFVHKTKGQI

SEQ ID NO: 50 - protein, 34 aa, Artificial Sequence
  artificial ANGPTL3 peptide
  Xaa (12)..(12): Xaa is Aib alpha-Amino-isobutyric acid
RFAMLDDVKILXNGLLQLGHGLKDFVHKTKGQIC

SEQ ID NO: 51 - protein, 34 aa, Artificial Sequence
  artificial ANGPTL3 peptide
  Xaa (13)..(13): Xaa is Aib alpha-Amino-isobutyric acid
CRFAMLDDVKILXNGLLQLGHGLKDFVHKTKGQI

SEQ ID NO: 52 - protein, 33 aa, Artificial Sequence
  artificial ANGPTL3 peptide
  Xaa (3)..(3): Xaa is AC3C 1-aminocyclopropane
RFXMLDDVKILANGLLQLGHGLKDFVHKTKGQI

SEQ ID NO: 53 - protein, 33 aa, Artificial Sequence
  artificial ANGPTL3 peptide
  Xaa (12)..(12): Xaa is AC3C- 1-aminocyclopropane
RFAMLDDVKILXNGLLQLGHGLKDFVHKTKGQI

SEQ ID NO: 54 - protein, 33 aa, Artificial Sequence
  artificial ANGPTL3 peptide
  Xaa (1)..(1): Xaa is Arg(NO2)  Arginine (NO2)
  Xaa (3)..(3): Xaa is AC5C- 1-aminocyclopentane
XFXMLDDVKILANGLLQLGHGLKDFVHKTKGQI

SEQ ID NO: 55 - protein, 33 aa, Artificial Sequence
  artificial ANGPTL3 peptide
  Xaa (3)..(3): Xaa is Aib alpha-Amino-isobutyric acid
  Xaa (6)..(6): Xaa is alpha-Me-Asp alpha-methyl-aspartic acid
RFXMLXDVKILANGLLQLGHGLKDFVHKTKGQI

SEQ ID NO: 56 - protein, 33 aa, Artificial Sequence
  artificial ANGPTL3 peptide
  Xaa (2)..(2): Xaa is alpha-Me-2,6-diF-Phe alpha-methyl-2,6-diflurophenylalanine
  Xaa (11)..(11): Xaa is HoLeu homoleucine
  Xaa (25)..(25): Xaa is alpha-Me-2,6-diF-Phe alpha-methyl-2,6-diflurophenylalanine
RXAMLDDVKIXANGLLQLGHGLKDXVHKTKGQI

SEQ ID NO: 57 - protein, 33 aa, Artificial Sequence
  artificial ANGPTL3 peptide
  Xaa (3)..(3): Xaa is AC3C-OH 1-aminocyclopropane-1- carboxylic acid
  Xaa (14)..(14): Xaa is beta-Ala beta alanine
  Xaa (25)..(25): Xaa is alpha-Me-2,6-diF-Phe alpha-methyl-2,6-diflurophenylalanine
RFXMLDDVKILANXLLQLGHGLKDXVHKTKGQI

SEQ ID NO: 58 - protein, 33 aa, Artificial Sequence
  artificial ANGPTL3 peptide
  Xaa (3)..(3): Xaa is Aib alpha-Amino-isobutyric acid
  Xaa (29)..(29): Xaa is Thr(OMe)  O-methyl-threonine
RFXMLDDVKILANGLLQLGHGLKDFVHKXKGQI

SEQ ID NO: 59 - protein, 33 aa, Artificial Sequence
  artificial ANGPTL3 peptide
  Xaa (3)..(3): Xaa is Aib alpha-Amino-isobutyric acid
  Xaa (14)..(14): Xaa is beta Ala beta alanine
  Xaa (29)..(29): Xaa is Thr(OMe)  O-methyl-threonine
RFXMLDDVKILANXLLQLGHGLKDFVHKXKGQI

SEQ ID NO: 60 - protein, 37 aa, Artificial Sequence
  artificial ANGPTL3 peptide
  Xaa (7)..(7): Xaa is Aib alpha-Amino-isobutyric acid
EPKSRFXMLDDVKILANGLLQLGHGLKDFVHKTKGQI

SEQ ID NO: 61 - protein, 37 aa, Artificial Sequence
  artificial ANGPTL3 peptide
  Xaa (16)..(16): Xaa is Aib alpha-Amino-isobutyric acid
EPKSRFAMLDDVKILXNGLLQLGHGLKDFVHKTKGQI

SEQ ID NO: 62 - protein, 37 aa, Artificial Sequence
  artificial ANGPTL3 peptide
  Xaa (5)..(5): Xaa is Arg(NO2)  Arginine (NO2)
  Xaa (7)..(7): Xaa is Aib alpha-Amino-isobutyric acid
EPKSXFXMLDDVKILANGLLQLGHGLKDFVHKTKGQI

SEQ ID NO: 63 - protein, 37 aa, Artificial Sequence; identical to SEQ ID NO: 62
  artificial ANGPTL3 peptide
  Xaa (5)..(5): Xaa is Arg(NO2)  Arginine (NO2)
  Xaa (7)..(7): Xaa is AC6C- 1-aminocyclohexane-

SEQ ID NO: 64 - protein, 37 aa, Artificial Sequence
  artificial ANGPTL3 peptide
  Xaa (5)..(5): Xaa is Arg(NO2)  Arginine (NO2)
  Xaa (7)..(7): Xaa is AC6C- 1-aminocyclohexane
  Xaa (18)..(18): Xaa is beta-Ala beta alanine
  Xaa (29)..(29): Xaa is alpha-Me-2,6-diF-Phe alpha-methyl-2,6-diflurophenylalanine
EPKSXFXMLDDVKILANXLLQLGHGLKDXVHKTKGQI

SEQ ID NO: 65 - protein, 43 aa, Artificial Sequence
  artificial ANGPTL3 peptide
SLSPEPKSRFAMLDDVKILANGLLQLGHGLKDFVHKTKGQIND

SEQ ID NO: 66 - protein, 43 aa, Artificial Sequence
  artificial ANGPTL3 peptide
  Xaa (11)..(11): Xaa is Aib alpha-Amino-isobutyric acid
SLSPEPKSRFXMLDDVKILANGLLQLGHGLKDFVHKTKGQIND

SEQ ID NO: 67 - protein, 43 aa, Artificial Sequence
  artificial ANGPTL3 peptide
  X (31)..(31): X is Cit Citrulline
SLSPEPKSRFAMLDDVKILANGLLQLGHGLXDFVHKTKGQIND

SEQ ID NO: 68 - protein, 43 aa, Artificial Sequence
  artificial ANGPTL3 peptide
  Xaa (33)..(33): Xaa is alpha-Me-2,6-diF-Phe alpha-methyl-2,6-diflurophenylalanine
SLSPEPKSRFAMLDDVKILANGLLQLGHGLKDXVHKTKGQIND

SEQ ID NO: 69 - protein, 43 aa, Artificial Sequence
  artificial ANGPTL3 peptide
  Xaa (19)..(19): Xaa is Nle Norleucine
SLSPEPKSRFAMLDDVKIXANGLLQLGHGLKDFVHKTKGQIND

SEQ ID NO: 70 - protein, 26 aa, Artificial Sequence
  artificial ANGPTL3 peptide
EPKSRFAMLDDVKILANGLLQLGHGL

SEQ ID NO: 71 - protein, 26 aa, Artificial Sequence
  artificial ANGPTL3 peptide
  Xaa (7)..(7): Xaa is Aib alpha-Amino-isobutyric acid
EPKSRFXMLDDVKILANGLLQLGHGL

SEQ ID NO: 72 - protein, 26 aa, Artificial Sequence
  artificial ANGPTL3 peptide
  Xaa (23)..(23): Xaa is Sar Sarcosine
EPKSRFAMLDDVKILANGLLQLXHGL

SEQ ID NO: 73 - protein, 26 aa, Artificial Sequence
  artificial ANGPTL3 peptide
  Xaa (13)..(13): Xaa is Cit Citrulline
EPKSRFAMLDDVXILANGLLQLGHGL

SEQ ID NO: 74 - protein, 26 aa, Artificial Sequence
  artificial ANGPTL3 peptide
  Xaa (12)..(12): Xaa is Nva Norvaline
EPKSRFAMLDDXKILANGLLQLGHGL

SEQ ID NO: 75 - protein, 33 aa, Artificial Sequence
  artificial ANGPTL3 peptide
  Xaa (3)..(3): Xaa is Aib alpha-Amino-isobutyric acid
  Xaa (12)..(12): Xaa is Aib alpha-Amino-isobutyric acid
RFXMLDDVKILXNGLLQLGHGLKDFVHKTKGQI

SEQ ID NO: 76 - protein, 33 aa, Artificial Sequence; identical to SEQ ID NO: 55
  artificial ANGPTL3 peptide
  Xaa (3)..(3): Xaa is AC5C- 1-aminocyclopentane
  Xaa (6)..(6): Xaa is alpha-Me-Asp alpha-methyl-aspartic acid

SEQ ID NO: 77 - protein, 33 aa, Artificial Sequence
  artificial ANGPTL3 peptide
  Xaa (3)..(3): Xaa is Aib alpha-Amino-isobutyric acid
  Xaa (31)..(31): Xaa is beta-Ala beta alanine
RFXMLDDVKILANGLLQLGHGLKDFVHKTKXQI

SEQ ID NO: 78 - protein, 33 aa, Artificial Sequence; identical to SEQ ID NO: 52
  artificial ANGPTL3 peptide
  Xaa (3)..(3): Xaa is Aib alpha-Amino-isobutyric acid

SEQ ID NO: 79 - protein, 33 aa, Artificial Sequence
  artificial ANGPTL3 peptide
  Xaa (1)..(1): Xaa is Cit Citrulline
  Xaa (9)..(9): Xaa is Cit Citrulline
XFAMLDDVXILANGLLQLGHGLKDFVHKTKGQI

SEQ ID NO: 80 - protein, 33 aa, Artificial Sequence
  artificial ANGPTL3 peptide
  Xaa (1)..(1): Xaa is Har homoarginine
XFAMLDDVKILANGLLQLGHGLKDFVHKTKGQI

SEQ ID NO: 81 - protein, 33 aa, Artificial Sequence
  artificial ANGPTL3 peptide
  Xaa (1)..(1): Xaa is Cit Citrulline
XFAMLDDVLILANGLLQLGHGLKDFVHKTKGQI

SEQ ID NO: 82 - protein, 34 aa, Artificial Sequence
  artificial ANGPTL3 peptide
  Xaa (2)..(2): Xaa is Cit Citrulline
  Xaa (10)..(10): Xaa is Cit Citrulline
CXFAMLDDVXILANGLLQLGHGLKDFVHKTKGQI

SEQ ID NO: 83 - protein, 34 aa, Artificial Sequence
  artificial ANGPTL3 peptide
  Xaa (2)..(2): Xaa is Har homoarginine
CXFAMLDDVKILANGLLQLGHGLKDFVHKTKGQI

SEQ ID NO: 84 - protein, 34 aa, Artificial Sequence
  artificial ANGPTL3 peptide
  Xaa (4)..(4): Xaa is Aib alpha-Amino-isobutyric acid
CRFXMLDDVKILANGLLQLGHGLKDFVHKTKGQI

SEQ ID NO: 85 - protein, 34 aa, Artificial Sequence
  artificial ANGPTL3 peptide
  Xaa (2)..(2): Xaa is Cit Citrulline
CXFAMLDDVLILANGLLQLGHGLKDFVHKTKGQI

SEQ ID NO: 86 - protein, 34 aa, Artificial Sequence
  artificial ANGPTL3 peptide
  Xaa (3)..(3): Xaa is APPA 2-Aminophenyl pentanoic acid
CRXAMLDDVKILANGLLQLGHGLKDFVHKTKGQI

SEQ ID NO: 87 - protein, 34 aa, Artificial Sequence
  artificial ANGPTL3 peptide
  Xaa (10)..(10): Xaa is Cit Citrulline
CRFAMLDDVXILANGLLQLGHGLKDFVHKTKGQI

SEQ ID NO: 88 - protein, 34 aa, Artificial Sequence
  artificial ANGPTL3 peptide
  Xaa (10)..(10): Xaa is Cit Citrulline
  Xaa (13)..(13): Xaa is Aib alpha-Amino-isobutyric acid
CRFAMLDDVXILXNGLLQLGHGLKDFVHKTKGQI

SEQ ID NO: 89 - protein, 34 aa, Artificial Sequence
  artificial ANGPTL3 peptide
  Xaa (6)..(6): Xaa is Nle Norleucine
  Xaa (10)..(10): Xaa is Cit Citrulline
CRFAMXDDVXILANGLLQLGHGLKDFVHKTKGQI

SEQ ID NO: 90 - protein, 34 aa, Artificial Sequence
  artificial ANGPTL3 peptide
  Xaa (4)..(4): Xaa is Aib alpha-Amino-isobutyric acid
  Xaa (10)..(10): Xaa is Cit Citrulline
CRFXMLDDVXILANGLLQLGHGLKDFVHKTKGQI

SEQ ID NO: 91 - protein, 34 aa, Artificial Sequence
  artificial ANGPTL3 peptide
  Xaa (3)..(3): Xaa is APPA 2-Aminophenyl pentanoic acid
  Xaa (10)..(10): Xaa is Cit Citrulline
CRXAMLDDVXILANGLLQLGHGLKDFVHKTKGQI

SEQ ID NO: 92 - protein, 34 aa, Artificial Sequence; identical to SEQ ID NO: 82
  artificial ANGPTL3 peptide
  Xaa (2)..(2): Xaa is Har homoarginine
  Xaa (10)..(10): Xaa is Cit Citrulline

SEQ ID NO: 93 - protein, 34 aa, Artificial Sequence
  artificial ANGPTL3 peptide
  Xaa (4)..(4): Xaa is Aib alpha-Amino-isobutyric acid
  Xaa (10)..(10): Xaa is Cit Citrulline
  Xaa (13)..(13): Xaa is Aib alpha-Amino-isobutyric acid
CRFXMLDDVXILXNGLLQLGHGLKDFVHKTKGQI

SEQ ID NO: 94 - protein, 34 aa, Artificial Sequence
  artificial ANGPTL3 peptide
  Xaa (2)..(2): Xaa is Har homoarginine
  Xaa (4)..(4): Xaa is Aib alpha-Amino-isobutyric acid
  Xaa (10)..(10): Xaa is Cit Citrulline
  Xaa (13)..(13): Xaa is Aib alpha-Amino-isobutyric acid
CXFXMLDDVXILXNGLLQLGHGLKDFVHKTKGQI

SEQ ID NO: 95 - protein, 34 aa, Artificial Sequence
  artificial ANGPTL3 peptide
  Xaa (2)..(2): Xaa is Har homoarginine
  Xaa (4)..(4): Xaa is Aib alpha-Amino-isobutyric acid
  Xaa (6)..(6): Xaa is Nle Norleucine
  Xaa (10)..(10): Xaa is Cit Citrulline
  Xaa (13)..(13): Xaa is Aib alpha-Amino-isobutyric acid
CXFXMXDDVXILXNGLLQLGHGLKDFVHKTKGQI

SEQ ID NO: 96 - protein, 34 aa, Artificial Sequence
  artificial ANGPTL3 peptide
  Xaa (9)..(9): Xaa is Cit Citrulline
  Xaa (12)..(12): Xaa is Aib alpha-Amino-isobutyric acid
RFAMLDDVXILXNGLLQLGHGLKDFVHKTKGQIC

SEQ ID NO: 97 - protein, 34 aa, Artificial Sequence
  artificial ANGPTL3 peptide
  Xaa (5)..(5): Xaa is Nle Norleucine
  Xaa (9)..(9): Xaa is Cit Citrulline
RFAMXDDVXILANGLLQLGHGLKDFVHKTKGQIC

SEQ ID NO: 98 - protein, 34 aa, Artificial Sequence
  artificial ANGPTL3 peptide
  Xaa (3)..(3): Xaa is Aib alpha-Amino-isobutyric acid
  Xaa (9)..(9): Xaa is Cit Citrulline
RFXMLDDVXILANGLLQLGHGLKDFVHKTKGQIC

SEQ ID NO: 99 - protein, 34 aa, Artificial Sequence
  artificial ANGPTL3 peptide
  Xaa (2)..(2): Xaa is APPA 2-Aminophenyl pentanoic acid
  Xaa (9)..(9): Xaa is Cit Citrulline
RXAMLDDVXILANGLLQLGHGLKDFVHKTKGQIC

SEQ ID NO: 100 - protein, 34 aa, Artificial Sequence
  artificial ANGPTL3 peptide
  Xaa (1)..(1): Xaa is Har homoarginine
  Xaa (9)..(9): Xaa is Cit Citrulline
XFAMLDDVXILANGLLQLGHGLKDFVHKTKGQIC

SEQ ID NO: 101 - protein, 34 aa, Artificial Sequence
  artificial ANGPTL3 peptide
  Xaa (3)..(3): Xaa is Aib alpha-Amino-isobutyric acid
  Xaa (9)..(9): Xaa is Cit Citrulline
  Xaa (12)..(12): Xaa is Aib alpha-Amino-isobutyric acid
RFXMLDDVXILXNGLLQLGHGLKDFVHKTKGQIC

SEQ ID NO: 102 - protein, 34 aa, Artificial Sequence
  artificial ANGPTL3 peptide
  Xaa (1)..(1): Xaa is Har homoarginine
  Xaa (3)..(3): Xaa is Aib alpha-Amino-isobutyric acid
  Xaa (9)..(9): Xaa is Cit Citrulline
  Xaa (12)..(12): Xaa is Aib alpha-Amino-isobutyric acid
XFXMLDDVXILXNGLLQLGHGLKDFVHKTKGQIC

SEQ ID NO: 103 - protein, 34 aa, Artificial Sequence
  artificial ANGPTL3 peptide
  Xaa (1)..(1): Xaa is Har homoarginine
  Xaa (3)..(3): Xaa is Aib alpha-Amino-isobutyric acid
  Xaa (5)..(5): Xaa is Nle Norleucine
  Xaa (9)..(9): Xaa is Cit Citrulline
  Xaa (12)..(12): Xaa is Aib alpha-Amino-isobutyric acid
XFXMXDDVXILXNGLLQLGHGLKDFVHKTKGQIC

SEQ ID NO: 104 - protein, 37 aa, Artificial Sequence; identical to SEQ ID NO: 60
  artificial ANGPTL3 peptide
  Xaa (7)..(7): Xaa is Aib alpha-Amino-isobutyric acid

SEQ ID NO: 105 - protein, 37 aa, Artificial Sequence; identical to SEQ ID NO: 61
  artificial ANGPTL3 peptide
  Xaa (16)..(16): Xaa is Aib alpha-Amino-isobutyric acid

SEQ ID NO: 106 - protein, 33 aa, Artificial Sequence; identical to SEQ ID NO: 75
  artificial ANGPTL3 peptide
  Xaa (3)..(3): Xaa is Aib alpha-Amino-isobutyric acid
  Xaa (12)..(12): Xaa is Aib alpha-Amino-isobutyric acid

SEQ ID NO: 107 - protein, 33 aa, Artificial Sequence; identical to SEQ ID NO: 55
  artificial ANGPTL3 peptide
  Xaa (3)..(3): Xaa is AC5C- 1-aminocyclopentane-
  Xaa (6)..(6): Xaa is alpha-Me-Asp alpha-methyl-aspartic acid

SEQ ID NO: 108 - protein, 33 aa, Artificial Sequence; identical to SEQ ID NO: 77
  artificial ANGPTL3 peptide
  Xaa (3)..(3): Xaa is Aib alpha-Amino-isobutyric acid
  Xaa (31)..(31): Xaa is beta-Ala beta alanine

SEQ ID NO: 109 - protein, 37 aa, Artificial Sequence; identical to SEQ ID NO: 62
  artificial ANGPTL3 peptide
  Xaa (5)..(5): Xaa is Arg(NO2)  Arginine (NO2)
  Xaa (7)..(7): Xaa is AC6C- 1-aminocyclohexane

SEQ ID NO: 110 - protein, 37 aa, Artificial Sequence; identical to SEQ ID NO: 64
  artificial ANGPTL3 peptide
  Xaa (5)..(5): Xaa is Arg(NO2)  Arginine (NO2)
  Xaa (7)..(7): Xaa is AC6C- 1-aminocyclohexane
  Xaa (18)..(18): Xaa is beta-Ala beta alanine
  Xaa (29)..(29): Xaa is al-Me-2,6-diF-Phe  alpha-methyl-2,6- diflurophenylalanine

SEQ ID NO: 111 - protein, 33 aa, Artificial Sequence
  artificial ANGPTL3 peptide
  Xaa (5)..(5): Xaa is Nle Norleucine
RFAMXDDVKILANGLLQLGHGLKDFVHKTKGQI